(19) United States
(12) Patent Application Publication
(10) Pub. No.: US 2014/0017174 A1
(43) Pub. Date: Jan. 16, 2014

(54) METHODS AND COMPOSITIONS FOR DETERMINING RESPONSIVENESS TO TREATMENT WITH A TNF-ALPHA INHIBITOR

(71) Applicants: Raja Atreya, Erlangen (DE); Markus F. Neurath, Erlangen (DE)

(72) Inventors: Raja Atreya, Erlangen (DE); Markus F. Neurath, Erlangen (DE)

(21) Appl. No.: 13/691,246

(22) Filed: Nov. 30, 2012

Related U.S. Application Data
(60) Provisional application No. 61/565,168, filed on Nov. 30, 2011.
(60) Provisional application No. 61/648,815, filed on May 18, 2012.

(51) Int. Cl. G01N 33/577; A61K 49/00

(52) U.S. Cl. CPC G01N 33/577; A61K 49/0058
USPC 424/9.6; 436/501; 424/158.1; 435/6.12; 506/9; 424/133.1; 424/145.1; 435/7.21

(57) ABSTRACT

The present invention is directed to methods and compositions useful for predicting the efficacy of a TNFα inhibitor for treating an inflammatory bowel disease (IBD). The invention includes, in one embodiment, determining the level of expression of TNFα by delivering a labeled anti-TNFα antibody on to the cells of the intestinal mucosa of a subject having IBD, whereby the TNFα level of expression can be used to predict whether the subject will be responsive or not to the antibody therapy. Levels of TNFα may be determined in vivo or ex vivo. The invention further provides methods of locally administering a TNFα antibody, e.g., topically to the intestinal mucosa, for the treatment of IBD.

### RELATED APPLICATIONS

This application claims priority to U.S. Provisional Patent Application No. 61/565,168, filed on Nov. 30, 2011, and U.S. Provisional Patent Application No. 61/648,815, filed on May 18, 2012. The entire contents of the priority applications are incorporated by reference herein in their entirety.

### BACKGROUND OF THE INVENTION

Treatment of inflammatory bowel disease (IBD) often depends on the form (e.g., Crohn's disease (CD) or ulcerative colitis), as well as on the extent and severity of the disease. Generally, depending on the level of severity, IBD may ultimately require systemic immunosuppression to control the symptoms, such as prednisone, azathioprine, methotrexate, 6-mercaptopurine or systemic tumor necrosis factor α (TNFα) inhibitors. Often, steroids are used to control disease flares. Topical therapy of IBD is generally limited to mild to moderate distal ulcerative colitis and can consist of mesalamine suppositories or enemas or hydrocortisone foam or enemas.

In recent years, treatment with systemic anti-TNFα antibodies has become a cornerstone for the therapy of CD and ulcerative colitis. This therapy binds and neutralizes an important mediator of inflammation, TNFα. Anti-TNFα antibodies are administered systemically, either intravenously or subcutaneously, and exert their effect via a systemic activity. The functional relevance of TNFα in CD is highlighted by the clinical efficacy of neutralizing anti-TNFα antibodies such as adalimumab, certolizumab pegol and infliximab (Colombel et al. N Engl J Med 362, 1383-1395 (2010); Evans and Lee, Expert Opin Biol Ther 12, 363-370 (2012); and Hanauer et al. 130, 323-333 (2006)). Anti-TNFα antibody therapy has been approved for treatment of patients with moderate to severe CD.

In spite of the clinical efficacy of anti-TNFα treatment, however, about 50% of patients do not respond to adalimumab treatment, as determined by a lacking 100 point reduction of the clinical activity score (CDAI) within 4 weeks after initiation of therapy (Hanauer et al. (2006) ibid). These patients demonstrate little or no improvement of clinical symptoms upon anti-TNFα therapy but are potentially exposed to undesired side effects of such treatment such as infections, allergic reactions, skin disorders and lupus-like autoimmunity (Colombel et al. Inflamm Bowel Dis 15, 1308-1319 (2009)). Thus, improved methods of treatment are needed.

### SUMMARY OF INVENTION

The instant invention provides unexpected results which solve both the problems of predicting which patients will be responsive to anti-TNFα therapy for treating an inflammatory bowel disease and providing improved methods of treatment. Applicants demonstrate that application of a labeled anti-TNFα antibody to cells of the intestinal mucosa in vivo or ex vivo can be used to determine the level of expression of membranous TNFα on the cells, and that the determined level of expression can be used to predict the subject's response to treatment with a TNFα inhibitor. As described herein, it has been determined that a high level of expression of mTNFα in the intestinal mucosa correlates with response to treatment with a TNFα inhibitor, and that a low level of expression of mTNFα in the intestinal mucosa correlates with non-response to treatment with a TNFα inhibitor. The methods described herein also relate to topical or intraluminal administration of therapeutic antibodies, including anti-TNFα antibodies, to a subject having an inflammatory bowel disease. Such local delivery provides an effective and safe method of treatment, while reducing systemic exposure. Accordingly, the present invention provides methods for determining the responsiveness of a subject having inflammatory bowel disease (IBD) to treatment with a TNFα inhibitor, as well as methods of localized treatment.

In one embodiment, the invention provides methods include determining the level of expression of TNFα in the cells of the intestinal mucosa of the subject having IBD, and comparing the level of expression of TNFα in the cells of the intestinal mucosa of the subject to a control level of expression of TNFα from a non-responder, wherein a higher level of expression of TNFα in the cells of the intestinal mucosa of the subject as compared to the control level of expression of TNFα indicates that the subject will be responsive to treatment with the TNFα inhibitor, thereby predicting the responsiveness of the subject having IBD to treatment with the TNFα inhibitor.

In another aspect, the invention provides a method for determining whether a TNFα inhibitor will be effective for the treatment of a subject having inflammatory bowel disease (IBD). The method includes determining the level of expression of TNFα in the cells of the intestinal mucosa of the subject having IBD, wherein a higher level of expression of TNFα in the cells of the intestinal mucosa of the subject as compared to a control level of expression of TNFα for a nonresponder indicates that the TNFα inhibitor will be effective for the treatment of the subject having IBD, thereby determining whether a TNFα inhibitor will be effective for the treatment of the subject having IBD.

In yet another aspect, the invention provides a method for treating a subject having inflammatory bowel disease (IBD). The method includes determining the level of expression of TNFα in the cells of the intestinal mucosa of the subject having IBD, and administering a TNFα inhibitor to the subject having IBD, provided that the level of expression of TNFα in the cells of the intestinal mucosa of the subject having IBD is higher than a control level of expression of TNFα for a nonresponder, thereby treating the subject having IBD.

In a further aspect, the invention methods of the invention are achieved by determining the level of expression of TNFα in the cells of the intestinal mucosa of the subject having IBD comprises topically applying a detectably labeled TNFα inhibitor to the cells of the intestinal mucosa of the subject having IBD. In one embodiment, the detectably labeled TNFα inhibitor is topically applied to the cells of the intestinal mucosa of the subject having IBD during colonoscopy.

In another aspect, the invention provides a method for treating a subject having inflammatory bowel disease (IBD). The method includes selecting a subject having IBD and having a level of expression of TNFα in the intestinal mucosa which is higher than a control level of expression of TNFα from a nonresponder, and topically administering a TNFα inhibitor to the intestinal mucosa of the subject having IBD, thereby treating the subject having IBD. In one embodiment, the TNFα inhibitor is administered using a spraying catheter.

Methods for determining responsiveness according to the invention may be achieved using in vivo or ex vivo assays.

In one embodiment of the invention, the level of expression of TNFα is determined using an in vivo assay. In one embodiment, the level of expression of TNFα is determined in vivo by confocal laser endomicroscopy. In one embodiment, a subject will be responsive to treatment of IBD with a TNFα inhibitor if the subject has twenty or more TNFα positive cells in an image obtained using endomicroscopy (e.g., a confocal laser endomicroscopy) that is about 475 μm×475 μm. In another, embodiment a subject will be responsive to treatment of IBD with a TNFα inhibitor if the subject has ten or more TNFα positive cells in an image obtained using endomicroscopy (e.g., a confocal laser endomicroscopy) that is about 240 μm×240 μm. In one embodiment, a subject will be responsive to treatment of IBD with a TNFα inhibitor if the subject has an increase of 180% in the number of TNFα positive cells an in vivo image in comparison to a non-responder control. Increases over 180%, e.g., 190%, 200%, 210%, 220%, 230%, 240%, etc. are also included in the methods of the invention, where, for example, a subject have a 230% increase in the image relative to a non-responder control would be determined to be responsive to treatment of IBD with a TNFα inhibitor.

In another embodiment of the invention, the level of expression of TNFα is determined using an ex vivo assay. For example, the level of expression of TNFα in the sample is determined by a technique selected from the group consisting of immunohistochemistry, immunocytochemistry, flow cytometry, ELISA and mass spectrometry. In another embodiment, the level of expression of TNFα in the sample is determined at the nucleic acid level, e.g., using either quantitative polymerase chain reaction or expression array analysis. In one embodiment, a subject will be responsive to treatment of IBD with a TNFα inhibitor if the subject has an increase of 170% in the level of expression of TNFα using an ex vivo assay in comparison to a non-responder control. Increases over 170%, e.g., 180%, 190%, 200%, 210%, 220%, 230%, 240%, etc. are also included in the methods of the invention, where, for example, a subject have a 185% increase in the level of expression of TNFα in comparison to a non-responder control, would be determined to be responsive to treatment of IBD with a TNFα inhibitor.

In another aspect, the invention provides a kit for determining if a TNFα inhibitor will be effective for the treatment of a subject having inflammatory bowel disease (IBD). The kit involves a means for determining the level of expression of TNFα in the cells of the intestinal mucosa of the subject having IBD, and instructions for recommended treatment for the subject based on the level of expression of TNFα in the cells of the intestinal mucosa of the subject having IBD, wherein a higher level of expression of TNFα in the cells of the intestinal mucosa of the subject as compared to a control level of expression of TNFα from a nonresponder indicates that the TNFα inhibitor will be effective for the treatment of the subject having IBD.

In one embodiment, the kit of the invention includes a pharmaceutical composition comprising the TNFα inhibitor. In another embodiment, the kit means for determining the level of expression of TNFα in the cells of the intestinal mucosa of the subject having IBD comprises a detectably labeled anti-TNFα antibody, or antigen-binding portion thereof, In one embodiment, the detectably labeled anti-TNFα antibody, or antigen-binding portion thereof, is labeled with fluorescein isothiocyanate (FITC). In one embodiment, the means for determining the level of expression of TNFα in the cells is a means for determining the level of membrane TNFα in the cells of the intestinal mucosa.

In one aspect of the invention, the IBD is Crohn's disease or ulcerative colitis.

In another aspect of the invention, the level of expression of membrane TNFα (mTNFa) in the cells of the intestinal mucosa of the subject having IBD is determined.

In one embodiment, the method of the invention determines or predicts clinical responsiveness in the subject.

In one embodiment, the methods and compositions of the invention include a TNFα inhibitor which is an anti-TNFα antibody, or antigen-binding portion thereof. In one embodiment, the anti-TNFα antibody, or antigen-binding portion thereof, is selected from the group consisting of a human antibody, a chimeric antibody, and a humanized antibody. In another embodiment, the chimeric anti-TNFα antibody, or antigen-binding portion thereof, is infliximab. In yet another embodiment, the human anti-TNFα antibody, or antigen-binding portion thereof, is adalimumab or golimumab. In one embodiment, the human anti-TNFα antibody, or antigen-binding portion thereof, is an isolated human antibody that dissociates from human TNFα with a K_{d} of 1×10⁻⁸ M or less and a k_{off} rate constant of 1×10⁻³ S⁻¹ or less, both determined by surface plasmon resonance, and neutralizes human TNFα cytotoxicity in a standard in vitro L929 assay with an IC₅₀ of 1×10⁻⁷ M or less. In one embodiment, the human anti-TNFα antibody, or antigen-binding portion thereof, is an isolated human antibody with the following characteristics: dissociates from human TNFα with a k_{off} rate constant of 1×10⁻³ s⁻¹ or less, as determined by surface plasmon resonance; has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9; and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12. In another embodiment, the human anti-TNFα antibody, or antigen-binding portion thereof, is an isolated human antibody with a light chain variable region (LCVR) comprising the amino acid sequence of SEQ ID NO: 1 and a heavy chain variable region (HCVR) comprising the amino acid sequence of SEQ ID NO: 2.

### BRIEF DESCRIPTION OF THE DRAWINGS

FIG. 1 describes ex vivo molecular imaging of mTNFα in surgical gut specimens from CD patients using fluorescent adalimumab. FIG. 1A depicts ex vivo molecular imaging of mTNFα in surgically resected gut specimens from CD patients which were incubated with fluorescent adalimumab to mimic topical application during endoscopy. Specific signals for mTNFα are indicated by arrows and single crypts with crypt lumina are within the circles. One representative experiment out of 5 is shown. FIG. 1B depicts confocal microscopy of gut cryosection with mTNFα expressing immune cells (arrows) from the same patients upon immunohistochemical staining with fluorescent adalimumab. One representative experiment out of five is shown.

FIG. 2 provides in vivo and ex vivo molecular imaging of mTNFα positive mucosal immune cells in the gut of CD patients. FIG. 2A depicts in vivo specific signals for mTNFα positive mucosal cells (arrows) upon topical administration of fluorescent adalimumab to the inflamed gut of a CD patient. One representative image from 25 CD patients is shown (×1000 magnification). FIG. 2B is an image showing molecular imaging of single mTNFα positive cells (arrows) in mucosa below crypts in CD patients (obtained by digital postprocessing of confocal in vivo images). FIG. 2C provides a high magnification image of a single mTNFα positive cell in the lamina propria of a CD patient upon topical administration of fluorescent adalimumab in vivo (×1000). FIG. 2C revealed the membranous fluorescence pattern of the mTNFα positive cell. Membranous cell staining of mTNFα in mucosal immune cells was comparable to the images obtained by molecular imaging in vivo. Quantitative analysis of ex vivo staining demonstrated that patients with clinical response to adalimumab therapy after 12 weeks had a significantly higher number of mTNFα expressing cells (mean number of 24 mTNFα expressing cells/high power field) than patients without clinical response (mean number of 13 mTNFα expressing cells/high power field). These results were statistically significant (Mean values±s.e.m.; *p=0.02) (FIG. 2D).

FIG. 3 provides clinical findings upon adalimumab treatment and in vivo molecular imaging of mTNFα-positive mucosal immune cells in CD. FIG. 3A depicts in vivo molecular imaging of low (left panel) and high (right panel) numbers of mTNFα expressing immune cells in the inflamed intestinal mucosa of CD patients. Images represent one quarter of full scale confocal endomicroscopic images (475 μm×475 μm). FIG. 3B shows the mean mTNFa-positive cells in relation to whether or not a patient responded to adalimumab therapy. Data represent mean values±s.e.m.; *p=0.00003. FIG. 3C depicts the mean histological inflammatory score of sections from mucosal biopsies from the area where molecular imaging in vivo was performed. Inflammation in these histological sections were blinded and graded by a pathologist with values ranging from 0 (no inflammation) to 3 (high inflammation). Data represent mean values±s.e.m.; n.s. not significant. FIG. 3D graphically depicts the clinical response (defined as a reduction of the CDAI score by ≧100 points) after 12 weeks of adalimumab treatment. Response rates are shown for all CD patients in the study (n=25) as well as for the patients with low (n=13) and high (n=12) mTNFα expression. Patients in the high mTNFα group showed a markedly higher response rate as compared to the group with low numbers of mTNFα positive cells.

FIG. 4 FIG. 4A provides a SDS gel electrophoresis of fluorescein labelled adalimumab (left panel is UV light exposure and right panel is Coomassie staining). (H) represents adalimumab and (HF1) represents fluorescein isothiocynate-adalimumab. FIG. 4B provides a hypothetical model of fluorescent adalimumab based on the analysis provided in FIG. 4A.

FIG. 5 describes clinical findings upon adalimumab therapy. FIG. 5A graphically depicts the clinical outcome analysis showing that CD patients with a higher number of mTNFα positive intestinal cells had a statistically significant reduction of their CDAI levels after 4 and 12 weeks of adalimumab treatment in comparison to the baseline CDAI before initiation of adalimumab therapy. Patients were subsequently followed over a period of 52 weeks. In the follow up of the patients with high mTNFα expression it was shown that this group has a sustained significant reduction of the CDAI score even one year after the initiation of the adalimumab treatment. In contrast, patients with low numbers of mTNFα positive cells did not show any significant reduction in CDAI scores. Data represent mean values±s.e.m.; *p=0.04; **p=0.02. ***p=0.006. FIG. 5B graphically depicts results showing that patients with high numbers of mTNFα expressing cells had a statistically significant reduction of their corticosteroid use after 4 and 12 weeks of adalimumab treatment in comparison to patients with low numbers of mTNFα expressing cells. Data represent mean values±s.e.m.; *p=0.04.

### DETAILED DESCRIPTION OF THE INVENTION

The instant invention provides solves both the problems of determining which patients will be responsive to an anti-TNFα therapy, and also providing improved methods of treatment.

In order that the present invention may be more readily understood, certain terms are first defined.

### I. DEFINITIONS

As used herein, the term “inflammatory bowel disease” or “IBD”, used interchangeably herein, refers to inflammatory conditions of the large and small intestine. Examples of an inflammatory bowel disease include, but are not limited to, Crohn's disease (also referred to herein as “CD”) and ulcerative colitis.

As used herein, the term “intestinal mucosa” refers to the lining of the intestines. The mucosa is the innermost layer of the gastrointestinal tract and surrounds the lumen, or open space, within the tube. In one embodiment, the intestinal mucosa includes the lining of the small intestine and the large intestine (which includes the cecum, colon, rectum and anal canal). In one embodiment, the intestinal mucosa includes the lining of the esophagus, stomach, small intestine and the large intestine.

As used herein, the term “expression”, refers to detecting transcription of the gene encoding tumor necrosis factor alpha (TNFα) or to detecting translation of TNFα protein. To detect expression of TNFα refers to the act of actively determining whether TNFα is expressed or not. To quantitate expression refers to the act of determining the level of TNFα, e.g., number of mTNFα positive cells. Detecting and/or quantitating expression can include determining whether TNFα expression is upregulated as compared to a control level, downregulated as compared to a control level, or substantially unchanged as compared to a control level. Therefore, the step of quantitating and/or detecting expression does not require that expression of TNFα actually is upregulated or downregulated, but rather, can also include detecting no expression of TNFα or detecting that the expression of TNFα has not changed or is not different (i.e., detecting no significant expression of TNFα or no significant change in expression of TNFα as compared to a control). In one embodiment, expression refers to detecting TNFα protein as it is found in the membrane of the cell (i.e., detecting mTNFa).

The term “level” or “amount” as used herein refers to the measurable quantity of TNFα. The amount may be either (a) an absolute amount as measured in an appropriate unit, e.g., number of cells, fluorescence intensity, molecules, moles or weight per unit volume or cell or (b) a relative amount. The level of expression of TNFα can be considered “high”, “low”, “increased” or “decreased” relative to a control level of expression or relative to the level of expression of TNFα in a “responder”, relative to either the level of expression of TNFα in a “non-responder”, or, in another embodiment, the level of expression of a subject who does not have an IBD. In one embodiment, the “level of expression” refers to the level of expression of mTNFα (e.g., the number of cells expressing mTNFα on their cell surface) in a sample from a subject or observed in the patient in vivo.

The term “control level” refers to an accepted or pre-determined level of TNFα which is used to compare the TNFα level derived from a sample of a patient or observed in the patient in vivo. In one embodiment, the control level is based on a subject(s) having IBD who responded to treatment with a TNFα inhibitor. In another embodiment, the control level indicates the TNFα level of an unaffected, i.e., non-disease, state of a subject who does not have IBD. In another embodiment, the control level indicates a subject or subjects having IBD who did not respond to treatment with a TNFα inhibitor, and, therefore, represents the disease state of a non-responder to anti-TNFα therapy. When compared to the control level of TNFα, deviation from the control level generally indicates either that the subject will be responsive to treatment of an IBD with a TNFα inhibitor or will not be responsive. Alternatively, when compared to the control level, equivalence to the control level generally indicates confirmation of responsiveness or lack thereof.

As used herein, “responder” includes, but is not limited to, a subject with IBD who has improved clinical disease status following treatment with a TNFα inhibitor (e.g., reduction in CDAI score or reduction in use of corticosteroids). In one embodiment, a responder is a subject having IBD who achieves a reduction of 100 points or more in their Crohn's Disease Activity Index (CDAI) score following treatment with a TNFα inhibitor. In one embodiment, a responder is a subject having IBD who achieves a reduction of 100 points or more in their Crohn's Disease Activity Index (CDAI) score in a specific time frame following treatment with a TNFα inhibitor. As used herein, “non-responder” includes, but is not limited to, a subject with IBD who has no, or limited improvement in their clinical disease status following treatment with a TNFα inhibitor (e.g., lack of reduction in CDAI score, lack of reduction in use of corticosteroids). In one embodiment, a non-responder is a subject having IBD who fails to achieve a reduction of 100 points or more in their Crohn's Disease Activity Index (CDAI) score following treatment with a TNFα inhibitor. In one embodiment, a non-responder is a subject having IBD who fails to achieve a reduction of 100 points or more in their Crohn's Disease Activity Index (CDAI) score in a specific time frame following treatment with a TNFα inhibitor.

The term “sample” as used herein refers to a collection or image of similar cells or tissue obtained from a subject. The source of the tissue or cell sample may be solid tissue as from a fresh, frozen and/or preserved organ or tissue sample or biopsy or aspirate. In a preferred embodiment, the sample is obtained from the intestinal mucosa of a subject. In one embodiment, the term “sample” includes an image of the intestinal mucosa from a subject.

The term “subject” or “patient,” as used herein interchangeably, refers to either a human or non-human animal. In one embodiment, the subject is a human.

As used herein, “TNFα” (or “TNFa”) is intended to refer to a human cytokine that exists as a 17 kD secreted form and a 26 kD membrane associated form (abbreviated here as “mTNFα”), the biologically active form of which is composed of a trimer of noncovalently bound 17 kD molecules. The structure of TNFα is described further in, for example Pennica et al. (1984) Nature 312: 724-729; Davis et al. (1987) Biochemistry 26:1322-1326; and Jones et al. (1989) Nature 338:225-228. The term TNFα is intended to include recombinant human TNFα (rhTNFa), which can be prepared by standard recombinant expression methods or purchased commercially (R & D Systems, Catalog No. 210-TA, Minneapolis, Minn.).

As used herein “mTNFa” (or “mTNFa”) refers to membrane TNFα. As used herein, “TNFα inhibitor” includes agents which inhibit TNFα. Examples of TNFα inhibitors include etancercept (ENBREL®, Immunex), infliximab (REMICADE®, Janssen/Johnson and Johnson), adalimumab (HUMIRA®, also referred to as D2E7, Abbott Laboratories), golimumab (SIMPONI®, Janssen/Johnson and Johnson), CDP 571 (Celltech), and certolizumab pegol (CIMZIA kor CDP 870 (Celltech) and other compounds which inhibit TNFα activity, such that when administered to a subject suffering from or at risk of suffering from a disorder in which TNFα activity is detrimental, the disorder is treated. The term also includes each of the anti-TNFα human antibodies and antibody portions described herein as well as those described in U.S. Pat. Nos. 6,090,382, 6,258,562, 6,509,015, and 7,223,394, each of which is incorporated by reference in its entirety.

As used herein, “detectably labeled TNFα inhibitor” refers to a TNFα inhibitor which is linked (e.g., covalently) to a molecule and can be used to determine the presence of the TNFα inhibitor. The detectably labeled TNFα inhibitor may be detected by the methods including, but not limited to, fluorescent, colormetric, spectrophotometric, optic, luminescent, radioactive, or X means.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Nonlimiting embodiments are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “antigen-binding portion” of an antibody (or simply “antibody portion” or “antibody fragment”), as used herein, refers to one or more fragments of an antibody that retain the ability to specifically bind to an antigen (e.g., hIL-13). It has been shown that the antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

An “isolated antibody”, as used herein, is intended to refer to an antibody that is substantially free of other antibodies having different antigenic specificities (e.g., an isolated antibody that specifically binds hTNFα is substantially free of antibodies that specifically bind antigens other than hTNFα). An isolated antibody that specifically binds hTNFα may, however, have cross-reactivity to other antigens, such as TNFα molecules from other species (discussed in further detail below). Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). In one embodiment, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jonsson et al. (1993) Ann. Biol. Clin. 51:19; JOnsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

The term “nucleic acid molecule”, as used herein, is intended to include DNA molecules and RNA molecules. A nucleic acid molecule may be single-stranded or double-stranded, but preferably is double-stranded DNA.

The term “isolated nucleic acid molecule”, as used herein in reference to nucleic acids encoding antibodies or antibody portions (e.g., VH, VL, CDR3) that bind hTNFα, is intended to refer to a nucleic acid molecule in which the nucleotide sequences encoding the antibody or antibody portion are free of other nucleotide sequences encoding antibodies or antibody portions that bind antigens other than hTNFα, which other sequences may naturally flank the nucleic acid in human genomic DNA. Thus, for example, an isolated nucleic acid of the invention encoding a VH region of an anti-hTNFα antibody contains no other sequences encoding other VH regions that bind antigens other than hTNFα.

The term “dose,” as used herein, refers to an amount of TNFα inhibitor which is administered to a subject.

The term “multiple-variable dose” includes different doses of a TNFα inhibitor which are administered to a subject for therapeutic treatment. “Multiple-variable dose regimen” or “multiple-variable dose therapy” describe a treatment schedule which is based on administering different amounts of TNFα inhibitor at various time points throughout the course of treatment. Multiple-variable dose regimens are described in US Patent Application Publication No. 20060009385, which is incorporated by reference herein in its entirety.

The term “dosing”, as used herein, refers to the administration of a substance (e.g., an anti-TNFα antibody) to achieve a therapeutic objective (e.g., the treatment of IBD).

The terms “biweekly dosing regimen”, “biweekly dosing”, and “biweekly administration”, as used herein, refer to the time course of administering a substance (e.g., an anti-TNFα antibody) to a subject to achieve a therapeutic objective. The biweekly dosing regimen is not intended to include a weekly dosing regimen. Preferably, the substance is administered every 9-19 days, more preferably, every 11-17 days, even more preferably, every 13-15 days, and most preferably, every 14 days. Biweekly dosing is further described in US Patent Application Publication No. 20030235585, which is incorporated by reference herein in its entirety.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an anti-TNFα antibody and another drug. The other drug(s) may be administered concomitant with, prior to, or following the administration of an anti-TNFα antibody.

The term “kit” as used herein refers to a packaged product comprising components with which to determine the responsiveness of a subject to treatment of IBD with a TNFα inhibitor, e.g., a means for detecting m TNFα in the intestinal mucosa of a subject. In one embodiment, the kit further provides components for administering aTNFα antibody of the invention for treatment of IBD. The kit preferably comprises a box or container that holds the components of the kit. The box or container is affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention.

### II. METHODS OF THE INVENTION

An unmet need in the treatment of IBD is to establish predictive biomarkers for therapeutic responders in order to avoid exposure of non-responders to anti-TNFα therapy, thus decreasing morbidity in patients with a low likelihood of response and enhancing safety and cost effective use of this treatment. Although patients with elevated CRP-levels in the blood have demonstrated higher response rates to anti-TNFα treatment (Vermeire et al. Inflamm Bowel Dis 10, 661-665 (2004)), there is a need for additional specific biomarkers that allow the prediction of response to anti-TNFα therapy for inflammatory bowel diseases. Thus, the prediction of clinical responsiveness to anti-TNFα antibodies is a key clinical problem and approaches aiming at a better prediction of responsiveness will have positive effects on the therapeutic use of these substances. The instant invention provides unexpected results which solve the problem of how to predict which IBD patients will be responsive to anti-TNFα therapy. The instant invention also provides safe ways of delivering anti-TNFα antibodies to a subject having IBD though topical delivery, thus providing improved methods of treatment. In one embodiment, the anti-TNFα antibody is topically administered to a subject having IBD, e.g., Crohn's disease, where the subject was selected as being a responder to TNFα inhibitor therapy.

### Methods for Determining Responsiveness to Treatment

The invention provides methods for predicting or determining the responsiveness of a subject having IBD to treatment with a TNFα inhibitor. Thus, the invention provides methods for determining whether a TNFα inhibitor will be effective for the treatment of a subject having IBD. In one embodiment, these methods comprise determining the level of expression of TNFα in the cells of the intestinal mucosa of a subject having IBD and comparing the level of expression of TNFα in the cells of the intestinal mucosa of the subject to a control level of expression of TNFα.

The control level of TNFα that may be used to determine responsiveness of a subject may be the level of TNFα, e.g., mTNFα, in the intestinal mucosa of a responder or a non-responder. A higher level of expression of TNFα in the cells of the intestinal mucosa of the subject as compared to a control level of expression of TNFα of a non-responder indicates that the subject will be responsive to treatment with a TNFα inhibitor. In contrast, an equivalent or lower level of TNFα in the cells of the intestinal mucosa of the subject as compared to the control level of expression of TNFα which is that of a non-responder indicates that the subject will not be responsive to treatment with a TNFα inhibitor. In another alternative, the control level of expression of TNFα may be the level of expression of TNFα in the intestinal mucosa of a responder. In such a case, if the subject's level of TNFα is greater or equivalent to the control level, then the subject having IBD will be responsive to treatment with a TNFα inhibitor. If the subject's level of TNFα is less than the control level, however, where the control is from a responder, then that is indicative of the fact that the subject having IBD will not be responsive to treatment with a TNFα inhibitor. In one embodiment, levels of TNFα are determined by the number of mTNFα positive cells in a sample from the subject.

In one embodiment, the invention provides a method for determining the responsiveness of a subject having inflammatory bowel disease (IBD) to treatment with a TNFα inhibitor, the method comprising determining the level of expression of TNFα in the cells of the intestinal mucosa of the subject having IBD; and comparing the level of expression of TNFα in the cells of the intestinal mucosa of the subject to a control level of expression of TNFα from a non-responder, wherein a higher level of expression of TNFα in the cells of the intestinal mucosa of the subject as compared to the control level of expression of TNFα indicates that the subject will be responsive to treatment with the TNFα inhibitor, thereby predicting the responsiveness of the subject having IBD to treatment with the TNFα inhibitor.

In an alternative, the invention provides a method of determining whether a TNFα inhibitor will be effective for the treatment of a subject having inflammatory bowel disease (IBD), the method comprising determining the level of expression of TNFα in the cells of the intestinal mucosa of the subject having IBD, wherein a higher level of expression of TNFα in the cells of the intestinal mucosa of the subject as compared to a control level of expression of TNFα for a nonresponder indicates that the TNFα inhibitor will be effective for the treatment of the subject having IBD, thereby determining whether a TNFα inhibitor will be effective for the treatment of the subject having IBD.

In one embodiment, the level of expression may be determined by assessing the level of expression of TNFα in cells which do not appear to be involved with disease and by comparing the foregoing lower level of TNFα with the level of expression of TNFα in cells in an area with disease involvement. For example, when endoscopy or another medical procedure reveals the presence of IBD involvement in one portion of an organ, the lower level of expression of TNFα may be assessed using the non-affected portion of the organ, and this lower level of expression may be compared with the level of expression of TNFα in an affected portion (e.g., inflamed mucosa) of the organ.

The level of expression of TNFα may be assessed in a variety of ways. In one embodiment of the invention, the level of expression of membrane TNFα (mTNFα) in the cells of the intestinal mucosa of the subject having IBD is determined by counting the number of mTNFα positive cells in a sample from the subject. This assessment may be performed in vivo, e.g., using endomicroscopy, or ex vivo, e.g., using histology analysis of intestinal mucosa biopsy sample(s) from a subject.

An anti-TNFα antibody used in the detection methods of the invention may be labelled with a detectable agent suitable for either in vivo or ex vivo analysis. Useful detectable agents with which an antibody or antibody portion of the invention may be derivatized include fluorescent compounds for either in vivo or ex vivo analysis. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like for ex vivo analysis. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

In one embodiment of the invention, the level of expression of TNFα in the intestinal mucosa of a subject having IBD is determined using an in vivo assay. In vivo imaging may be used to determine whether a subject having IBD will be responsive to treatment with a TNFα inhibitor, e.g., an anti-TNFα antibody. Such imaging may be performed during a colonoscopy on the subject, e.g., a colonoscopy to determine the severity of the IBD. During the procedure, an anti-TNFα antibody may be delivered locally to the intestinal mucosa to determine TNFα expression. For example, a spray catheter may be used in conjunction with an endoscope (e.g., Glo-Tip Spray Catheter; Cook Medical) to topically deliver a TNFα inhibitor, e.g., an anti-TNFα antibody to the subject for analysis. Preferably, the antibody is detectably labeled, e.g., FITC-adalimumab. Following topical administration of the antibody, in vivo molecular imaging may be performed to determine the level of mTNFα expression in the mucosa of the subject. In one embodiment, levels of TNFα are determined according to the number of TNFα positive cells counted in a given image.

In order to determine the expression level of TNFα in the intestinal mucosa, a detectably labeled anti-TNFα antibody, or antigen-binding portion thereof, may be administered to the subject, for example, by using a spraying catheter. The labeled antibody, or antigen-binding portion thereof, may be delivered to the intestinal tract of the subject during a colonoscopy. In one embodiment, the anti-TNFα antibody, or antigen-binding portion thereof, is delivered to a mucosal site within the large intestine having inflammation. Following delivery, imaging may be performed according to standard methods known in the art. In one embodiment, imaging of the intestinal mucosa of the subject is performed using confocal laser endomicroscopy.

In one embodiment the level of expression of TNFα is determined by topically applying a detectably labeled TNFα inhibitor to the cells of the intestinal mucosa of a subject having IBD. In yet another embodiment, the detectably labeled TNFα inhibitor is labeled with fluorescein isothiocyanate.

Endoscopy has witnessed a rapid evolution of endoscopic techniques for improved detection of inflammatory and neoplastic lesions in recent years (Neumann et al. Gastroenterology 139, 388-392, 392 e381-382 (2010); Kendall et al. The Journal of pathology 200, 602-609 (2003); Evans et al. Gastrointestinal endoscopy 65, 50-56 (2007); Lovat et al. Gut 55, 1078-1083 (2006); Herrero et al. Gastroenterology Clinics of North America 39, 747-758 (2010); Qiu et al. Nat Med 16, 603-606, 601p following 606 (2010); and Waldner et al. Nat Protoc 6, 1471-1481 (2011)). In addition to filter techniques such as narrow band imaging, optical coherence tomography, Raman spectroscopy, elastic scattering spectroscopy and multispectral imaging have been introduced. Furthermore, confocal laser endomicroscopy has recently been shown to augment detection of local inflammation and neoplasia in the gastrointestinal tract by providing optical biopsies and in vivo imaging during ongoing endoscopy (Kiesslich et al. Gastroenterology 132, 874-882 (2007) and Kiesslich et al. Gut (2011)). For instance, endomicroscopy has been used in esophageal squamous cell carcinoma, Barrett's esophagus, colonic polyps, collagenous colitis and CD. In addition, endomicroscopy permitted the identification of neoplastic lesions during colonoscopy in patients by using a labelled heptapeptide derived from a phage library (Hsiung et al. Nat Med 14, 454-458 (2008)).

Thus, in vivo methods described herein may be accomplished using endomicroscopy, including confocal laser endomicroscopy. Examples of confocal laser endomicroscopes that may be used include the Pentax Endomicroscopy System (Pentax) and the Cellvizio high resolution confocal microscope (Mauna Kea Technologies).

In one embodiment, 20 or more TNFα positive cells in an in vivo image that is at least 475 μm×475 μm indicates that the subject will be responsive to treatment with an anti-TNFα antibody, or antigen-binding portion thereof. Alternatively, less than 20 TNFα positive cells in an in vivo image that is at least 475 μm×475 μm indicates that the subject will not be responsive to treatment with an anti-TNFα antibody, or antigen-binding portion thereof. Optical sections of 475 μm×475 μm can be obtained using a high resolution confocal microscope, such as, but not limited to, the Pentax endomicroscopic system (Pentax).

In another embodiment, 10 or more TNFα positive cells in an in vivo image that is at least 240 μm×240 μm indicates that the subject will be responsive to treatment with an anti-TNFα antibody, or antigen-binding portion thereof. Alternatively, less than 10 TNFα positive cells in an in vivo image that is at least 240 μm×240 μm indicates that the subject will not be responsive to treatment with an anti-TNFα antibody, or antigen-binding portion thereof. Optical sections of 240 μm×240 μm can be obtained using a high resolution confocal microscope, such as, but not limited to, the Cellvizio high resolution confocal microscope (Mauna Kea Technologies).

In one embodiment, at least a 180% increase (or at least a 185%, 190%, 195%, 200%, 205%, 210%, 215%, 220%, or 225%) in the level of expression of TNFα, e.g., the number of TNFα positive cells, in an in vivo image relative to the same size image from a non-responder control indicates that the subject will be responsive to treatment with an anti-TNFα antibody, or antigen-binding portion thereof. Alternatively, an equivalent or increased level of expression of TNFα, e.g., number of TNFα positive cells, in an in vivo image relative to the same size image from a responder control indicates that the subject will be responsive to treatment with an anti-TNFα antibody, or antigen-binding portion thereof. In one embodiment, an increase of 230%, 235%, 240%, 245%, 250%, 255%, 260%, 265%, 270%, 275%, 280%, 285%, 290%, 295%, or 300% in the level of expression of TNFα, e.g., number of TNFα positive cells, in an in vivo image relative to the same size image from a non-responder control indicates that the subject will be responsive to treatment with an anti-TNFα antibody, or antigen-binding portion thereof. The level of expression of TNFα, e.g., the number of TNFα positive cells may also be determined ex vivo using standard histology techniques, as described below.

The invention also provides methods of predicting the responsiveness of a subject having IBD to treatment with a TNFα inhibitor where the level of expression of TNFα is determined ex vivo. In ex vivo methods, the level of expression of TNFα in a sample of cells from the intestinal mucosa of a subject with IBD may be compared with sample of cells from a control (responder or non-responder). A lower level of expression of TNFα in the subject's sample, relative to a responder sample, is an indication that the subject will not respond to treatment with a TNFα inhibitor. A higher level of expression of TNFα in the subject's sample, relative to the non-responder sample, is an indication that that subject will respond to treatment with a TNFα inhibitor. Such a sample may be obtained by taking a biopsy from the mucosa of the intestinal tract of a subject having IBD.

Samples useful in the methods of invention for determining the level of TNFα expression include any tissue, cell, biopsy, or surgically resected sample from a subject having IBD that may express TNFα. Body samples for ex vivo analysis may be obtained from a subject using a variety of techniques know in the art including, for example, during a surgical procedure or by use of a biopsy or by scraping or swabbing an area. The samples may, for example, be obtained during a colonoscopy. In particular embodiments, the body sample comprises intestinal tissue samples. In one embodiment, the tissue sample is a small intestine tissue sample or a large intestine tissue sample.

In one embodiment, the level of expression of TNFα is detected on a protein level using, for example, antibodies that specifically bind TNFα. The level of TNFα expression may be determined by topically applying an anti-TNFα antibody, or antigen-binding portion thereof, to the intestinal mucosa of a subject having IBD, obtaining a sample from a biopsy of the intestinal mucosa on which the anti-TNFα antibody, or antigen-binding portion thereof, was applied, and assaying the sample for levels of expression of TNFα. The anti-TNFα antibody, or antigen-binding portion thereof, may be labelled with a detectable agent, e.g., FITC. Alternatively, the anti-TNFα antibody, or antigen-binding portion thereof, may not be labelled and may be assayed according to methods known in the art. In another embodiment, the sample is obtained via a biopsy from the intestinal mucosa of a subject having IBD, whereupon an anti-TNFα antibody, or antigen-binding portion thereof, is applied ex vivo to the sample for analysis of the expression level of TNFα.

In one embodiment, 15 or more (e.g., 16 or more, 17 or more, 18 or more, 19 or more, or 20 or more) TNFα positive cells in an image obtained from ex vivo analysis of an intestinal mucosa sample from a subject having IBD (for example, an image that is magnified by a SP-5 confocal microscope with a 63×/1.3 NA objective (Leica Microsystems)) indicates that the subject will be responsive to treatment with an anti-TNFα antibody, or antigen-binding portion thereof. Alternatively, less than 15 (e.g., 14 or less, 13 or less, etc.) TNFα positive cells in an in vivo image (for example an image that is at least magnified by a SP-5 confocal microscope with a 63×/1.3 NA objective (Leica Microsystems)) indicates that the subject will not be responsive to treatment with an anti-TNFα antibody, or antigen-binding portion thereof.

In one embodiment, a 170% increase in the level of TNFα expression, e.g., number of TNFα positive cells, in an image obtained from an ex vivo source, e.g., a histological section of the intestinal mucosa of a subject, relative to a control, e.g., an image obtained from an ex vivo source of a non-responder, indicates that the subject will be responsive. In one embodiment, an increase of 180% in the level of TNFα expression, e.g., the number of TNFα positive cells of a sample from a subject relative to a sample from a non-responder indicates that the subject will be responsive to treatment with a TNFα inhibitor. Increases of 185%, 190%, 195%, 200%, 205%, and so forth also indicate a likelihood of responsiveness in a subject. Alternatively, a 170% decrease in the levels of TNFα expression, e.g., number of TNFα positive cells, in an image obtained from an ex vivo source, e.g., a histological section of the intestinal mucosa of a subject, relative to a control, e.g., an image obtained from an ex vivo source of a responder, indicates that the subject will be not be responsive to TNFα therapy for treatment of IBD. Decreases of 185%, 190%, 195%, 200%, 205%, and so forth also indicate a likelihood of responsiveness in a subject.

Tissue samples suitable for ex vivo detecting and quantifying the level of expression of TNFα may be fresh, frozen, or fixed according to methods known to one of skill in the art. Suitable tissue samples are preferably sectioned and placed on a microscope slide for further analyses. Alternatively, solid samples, i.e., tissue samples, may be analyzed.

In one embodiment, a freshly obtained biopsy sample is frozen using, for example, liquid nitrogen or difluorodichloromethane. The frozen sample is mounted for sectioning using, for example, OCT, and serially sectioned in a cryostat. The serial sections are collected on a glass microscope slide. For immunohistochemical staining the slides may be coated with, for example, chrome-alum, gelatine or poly-L-lysine to ensure that the sections stick to the slides. In another embodiment, samples are fixed and embedded prior to sectioning. For example, a tissue sample may be fixed in, for example, formalin, serially dehydrated and embedded in, for example, paraffin.

Once the sample is obtained any method known in the art to be suitable for detecting and quantitating the level of expression of TNFα may be used (either at the nucleic acid or, preferably, at the protein level). Such methods are well known in the art and include but are not limited to western blots, northern blots, southern blots, immunohistochemistry, immunocytochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods.

Samples for ex vivo analysis may need to be modified in order to make the TNFα protein accessible to antibody binding. In a particular aspect of the immunocytochemistry or immunohistochemistry methods, slides may be transferred to a pretreatment buffer and optionally heated to increase antigen accessibility. Heating of the sample in the pretreatment buffer rapidly disrupts the lipid bi-layer of the cells and makes the antigens (may be the case in fresh specimens, but not typically what occurs in fixed specimens) (i.e., the TNFα) more accessible for antibody binding. The pretreatment buffer may comprise a pH-specific salt solution, a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffer may, for example, be a solution of 0.1% to 1% of deoxycholic acid, sodium salt, or a solution of sodium laureth-13-carboxylate (e.g., Sandopan LS) or and ethoxylated anionic complex. In some embodiments, the pretreatment buffer may also be used as a slide storage buffer. Any method for making TNFα protein more accessible for antibody binding may be used in the practice of the invention, including the antigen retrieval methods known in the art. See, for example, Bibbo, et al. (2002) Acta. Cytol. 46:25-29; Saqi, et al. (2003) Diagn. Cytopathol. 27:365-370; Bibbo, et al. (2003) Anal. Quant. Cytol. Histol. 25:8-11, the entire contents of each of which are incorporated herein by reference.

Following pretreatment to increase TNFα protein accessibility, samples may be blocked using an appropriate blocking agent, e.g., a peroxidase blocking reagent such as hydrogen peroxide. In some embodiments, the samples may be blocked using a protein blocking reagent to prevent non-specific binding of the antibody. The protein blocking reagent may comprise, for example, purified casein. An antibody, particularly a monoclonal antibody that specifically binds to TNFα is then incubated with the sample.

In one embodiment the level of expression of TNFα is determined by topically applying a detectably labeled TNFα inhibitor, e.g., an anti-TNFα antibody, to the cells of the intestinal mucosa of a subject having IBD. In yet another embodiment, the detectably labeled TNFα inhibitor is labeled with fluorescein isothiocyanate. Alternatively, the detectably labeled TNFα inhibitor, e.g., an anti-TNFα antibody, may be applied directly to a sample obtained from the subject, e.g., a tissue biopsy.

Techniques for ex vivo antibody detection are well known in the art. Antibody binding to TNFα may be detected through the use of chemical reagents that generate a detectable signal that corresponds to the level of antibody binding and, accordingly, to the level of TNFα protein expression. In one of the immunohistochemistry or immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include, but are not limited to, horseradish peroxidase (HRP) and alkaline phosphatase (AP).

In one particular immunohistochemistry or immunocytochemistry method of the invention, antibody binding to the TNFα proteins is detected through the use of an HRP-labeled polymer that is conjugated to a secondary antibody. Antibody binding can also be detected through the use of a species-specific probe reagent, which binds to monoclonal or polyclonal antibodies, and a polymer conjugated to HRP, which binds to the species specific probe reagent. Slides are stained for antibody binding using any chromagen, e.g., the chromagen 3,3-diaminobenzidine (DAB), and then counterstained with hematoxylin and, optionally, a bluing agent such as ammonium hydroxide or TBS/Tween-20. Other suitable chromagens include, for example, 3-amino-9-ethylcarbazole (AEC). In some aspects of the invention, slides are reviewed microscopically by a cytotechnologist and/or a pathologist to assess cell staining, e.g., fluorescent staining (i.e., TNFα expression). Alternatively, samples may be reviewed via automated microscopy or by personnel with the assistance of computer software that facilitates the identification of positive staining cells.

Detection of antibody binding can be facilitated by coupling the anti-TNFα antibodies to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, ¹⁴C, or ³H.

In one embodiment of the invention frozen samples are prepared as described above and subsequently stained with antibodies against TNFα diluted to an appropriate concentration using, for example, Tris-buffered saline (TBS). Primary antibodies can be detected by incubating the slides in biotinylated anti-immunoglobulin. This signal can optionally be amplified and visualized using diaminobenzidine precipitation of the antigen. Furthermore, slides can be optionally counterstained with, for example, hematoxylin, to visualize the cells.

In another embodiment, fixed and embedded samples are stained with antibodies against TNFα and counterstained as described above for frozen sections. In addition, samples may be optionally treated with agents to amplify the signal in order to visualize antibody staining. For example, a peroxidase-catalyzed deposition of biotinyl-tyramide, which in turn is reacted with peroxidase-conjugated streptavidin (Catalyzed Signal Amplification (CSA) System, DAKO, Carpinteria, Calif.) may be used.

One of skill in the art will recognize that the concentration of a particular antibody used to practice the methods of the invention will vary depending on such factors as time for binding, level of specificity of the antibody for TNFα, and method of sample preparation. Moreover, when multiple antibodies are used, the required concentration may be affected by the order in which the antibodies are applied to the sample, e.g., simultaneously as a cocktail or sequentially as individual antibody reagents. Furthermore, the detection chemistry used to visualize antibody binding to TNFα must also be optimized to produce the desired signal to noise ratio.

In one embodiment of the invention, proteomic methods, e.g., mass spectrometry, are used for detecting and quantitating the TNFα protein. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate the TNFα proteins. Mass spectrometric methods are described in, for example, U.S. Pat. Nos. 5,622,824, 5,605,798 and 5,547,835, the entire contents of each of which are incorporated herein by reference.

In other embodiments, the level of expression of TNFα is detected at the nucleic acid level. Nucleic acid-based techniques for assessing expression are well known in the art and include, for example, determining the level of TNFα mRNA in a body sample. Many expression detection methods use isolated RNA. Any RNA isolation technique that does not select against the isolation of mRNA can be utilized for the purification of RNA from cells that express TNFα (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155). In one embodiment, nucleic acids are analysed by either quantitative polymerase chain reaction or expression array analysis.

The term “probe” refers to any molecule that is capable of selectively binding to TNFα, for example, TNFα nucleotide transcript or TNFα protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the TNFα mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to TNFα mRNA or TNFα genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of TNFα mRNA.

An alternative method for determining the level of pTNFα mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-B eta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, TNFα expression is assessed by quantitative fluorogenic RT-PCR (i.e., the TaqMan™ System). Such methods typically utilize pairs of oligonucleotide primers that are specific for TNFα. Methods for designing oligonucleotide primers specific for a known sequence are well known in the art.

The expression levels of TNFα mRNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of TNFα expression may also comprise using nucleic acid probes in solution.

In one embodiment of the invention, microarrays are used to detect TNFα expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

### Methods of Treatment

The invention also provides methods for treating a subject having IBD. Examples of inflammatory bowel diseases that may be treated by the methods described herein include, but are not limited to, Crohn's disease and ulcerative colitis.

Crohn's disease (CD) represents one of the major entities of inflammatory bowel diseases, and is characterized by a chronic relapsing inflammation of the intestinal mucosa (Strober et al. J Clin Invest 117, 514-521 (2007) and Danese, S, New therapies for inflammatory bowel disease: from the bench to the bedside. Gut 61, 918-932 (2012)). Patients with this incurable disease suffer from chronic diarrhea, rectal bleeding, abdominal cramping and fistula formation and many patients require surgical intervention over time. It is general consensus that inappropriate activation of the mucosal immune system leading to augmented cytokine production contributes to disease pathogenesis (Neurath et al. Immunity 31, 357-361 (2009) and Atreya, et al. Nat Med 6, 583-588 (2000)). In this context, the pro-inflammatory cytokine tumor necrosis factor-α (TNFα) plays a pivotal role in CD immunopathogenesis. It is synthesised as a transmembrane protein (mTNFα) from which the soluble form (sTNFα) is released. While sTNFα preferentially binds to TNF receptor 1 on target cells, mTNFα mainly binds to TNF receptor 2. Intracellular TNFα signalling is mediated by members of the TNFR-associated family of regulatory proteins that lead to activation of the transcription factor NF-kappaB to induce pro-inflammatory immune responses in CD (Atreya et al. Gastroenterology 141, 2026-2038 (2011) and ten Hove et al. Gut 50, 206-211 (2002)).

Ulcerative colitis may also be treated by the methods disclosed herein. Ulcerative colitis is a type of inflammatory bowel disease (IBD) that affects the lining of the large intestine (colon) and rectum.

In one embodiment, a TNFα inhibitor, e.g., an anti-TNFα antibody, or antigen binding portion thereof, is administered topically to the intestinal mucosa of a subject having IBD for treatment. Topical administration may occur, for example, during a colonoscopy or during surgery.

The invention further provides a method of first determining the level of expression of TNFα in the cells of the intestinal mucosa of a subject having IBD and subsequently topically administering a TNFα inhibitor, e.g., an anti-TNFα antibody, or antigen binding portion thereof, to the subject having IBD for treatment, provided that the level of expression of TNFα in the cells of the intestinal mucosa of the subject having IBD is higher than a non-responder control level of expression of TNFα (or equal to or greater than a responder control level of expression). In one embodiment, the invention describes a method including selecting a subject having IBD and having a level of expression of TNFα in the intestinal mucosa which is higher than a non-responder control level of expression of TNFα (or equivalent to or higher than a responder level) and topically administering a TNFα inhibitor, e.g., an anti-TNFα antibody, or antigen binding portion thereof, to the intestinal mucosa of the subject having IBD.

Topical delivery of the TNFα inhibitor, e.g., anti-TNFα antibody, or antigen-binding portion thereof, to the intestinal mucosa may be achieved using methods known in the art. Topical delivery may be for diagnostic purposes, i.e., to determine if the subject will be responsive to an anti-TNFα antibody, or antigen-binding portion thereof, (as described above) or for therapeutic purposes, or both. Topical administration may occur, for example, during colonoscopy or during surgery.

In one embodiment, an anti-TNFα antibody, or antigen-binding portion thereof, may be administered to the intestinal mucosa of a subject having IBD using a spraying catheter.

Compositions for use in the methods and compositions of the invention may be in a variety of forms suitable for topical delivery to the intestinal mucosa. These include, for example, liquid, semi-solid and solid dosage forms, such as liquid solutions, dispersions or suspensions, tablets, pills, powders, liposomes and suppositories.

In certain embodiments, a TNFα inhibitor, e.g., anti-TNFα antibody, or antigen-binding portion thereof, may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The pharmaceutical composition used in the methods of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

In one embodiment, the subject having IBD who is identified as a responder to TNFα inhibitor therapy according to the methods described herein, is treated with a human anti-TNFα antibody, or antigen-binding portion thereof, according to a multiple variable dose regimen. Multiple-variable dose regimens are described in US Publication No. 20060009385, which is incorporated by reference herein in its entirety. In one embodiment, a subject identified as a responder is subcutaneously administered a loading or induction dose (s) followed by subsequent treatment or maintenance doses. In one embodiment, the subject is subcutaneously administered a first dose of 160 mg, a second dose of 80 mg, and a dose of 40 mg. In a further embodiment a dose of 80 mg is administered subcutaneously followed by a dose of 40 mg for treatment of IBD in a subject identified as a responder.

### III. TNFα INHIBITORS FOR USE IN INVENTION

The invention provides a method for determining the whether a subject will respond to treatment with a TNFα inhibitor, and, in some embodiments, topical delivery of the TNFα inhibitor in said subject.

In one embodiment, the TNFα inhibitor used in the methods and compositions of the invention is an anti-TNFα antibody, or antigen-binding portion thereof, such as, but not limited to, a human antibody, a chimeric antibody, and a humanized antibody. An example of a chimeric antibody that may be used is infliximab.

In one embodiment, the invention features uses and composition for predicting or determining the responsiveness of a subject having an IBD to treatment with a TNFα inhibitor, wherein the TNFα antibody is an isolated human antibody, or antigen-binding portion thereof, that binds to human TNFα with high affinity and a low off rate, and also has a high neutralizing capacity. Examples of such antibodies include adalimumab or golimumab. Preferably, the human antibodies used in the invention are recombinant, neutralizing human anti-hTNFα antibodies. The most preferred recombinant, neutralizing antibody of the invention is referred to herein as adalimumab, also referred to as HUMIRA® or D2E7(the amino acid sequence of the adalimumab VL region is shown in SEQ ID NO: 1; the amino acid sequence of the adalimumab VH region is shown in SEQ ID NO: 2; the nucleic acid sequence of the VL and VH domains are described in SEQ ID NOs: 36 and 37, respectively). The properties (and sequences) of D2E7 (adalimumab/HUMIRA®) have been described in Salfeld et al., U.S. Pat. Nos. 6,090,382, 6,258,562, and 6,509,015, which are each incorporated by reference herein.

In one embodiment, the TNFα inhibitor for use in the invention is a fully human TNFα antibody which is a biosimilar to adalimumab. In one embodiment, the TNFα inhibitor is highly similar to adalimumab, and may, for example, include minor differences in clinically inactive components. In one embodiment, the TNFα inhibitor is interchangeable with adalimumab, and is, for example, able to produce the same clinical result as adalimumab in any given patient.

In one embodiment, the method of the invention includes determining the responsiveness of a subject to treatment of IBD with adalimumab and antibody portions, adalimumab-related antibodies and antibody portions, or other human antibodies and antibody portions with equivalent properties to adalimumab, such as high affinity binding to hTNFα with low dissociation kinetics and high neutralizing capacity, for the treatment of an IBD, e.g., Crohn's disease. In one embodiment, the invention provides treatment with an isolated human antibody, or an antigen-binding portion thereof, that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a k_{off} of 5×10⁴ s⁻¹ or less, or even more preferably, with a k_{off} of 1×10⁴ s⁻¹ or less. More preferably, the isolated human antibody, or antigen-binding portion thereof, neutralizes human TNFα cytotoxicity in a standard in vitro L929 assay with an IC₅₀ of 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating an IBD, e.g., Crohn's disease, by administering human antibodies that have slow dissociation kinetics for association with hTNFα and that have light and heavy chain CDR3 domains that structurally are identical to or related to those of adalimumab. Position 9 of the adalimumab VL CDR3 can be occupied by Ala or Thr without substantially affecting the k_{off}. Accordingly, a consensus motif for the adalimumab VL CDR3 comprises the amino acid sequence: Q-R—Y—N—R-A-P—Y-(T/A) (SEQ ID NO: 3). Additionally, position 12 of the adalimumab VH CDR3 can be occupied by Tyr or Asn, without substantially affecting the k_{off}. Accordingly, a consensus motif for the adalimumab VH CDR3 comprises the amino acid sequence: V—S—Y-L-S-T-A-S—S-L-D-(Y/N) (SEQ ID NO: 4). Moreover, as demonstrated in Example 2 of U.S. Pat. No. 6,090,382, the CDR3 domain of the adalimumab heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the k_{off}. Still further, the skilled artisan will appreciate that, given the amenability of the adalimumab VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the adalimumab VL and/or VH CDR3 domains. More preferably, no more than one to three conservative amino acid substitutions are made within the adalimumab VL and/or VH CDR3 domains. Additionally, conservative amino acid substitutions should not be made at amino acid positions critical for binding to hTNFα. Positions 2 and 5 of the adalimumab VL CDR3 and positions 1 and 7 of the adalimumab VH CDR3 are critical for interaction with hTNFα and thus, conservative amino acid substitutions preferably are not made at these positions (although an alanine substitution at position 5 of the adalimumab VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a k_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a k_{off} of 5×10⁴ s⁻¹ or less. Even more preferably, the antibody, or antigen-binding portion thereof, dissociates from human TNFα with a k_{off} of 1×10⁴ s⁻¹ or less.

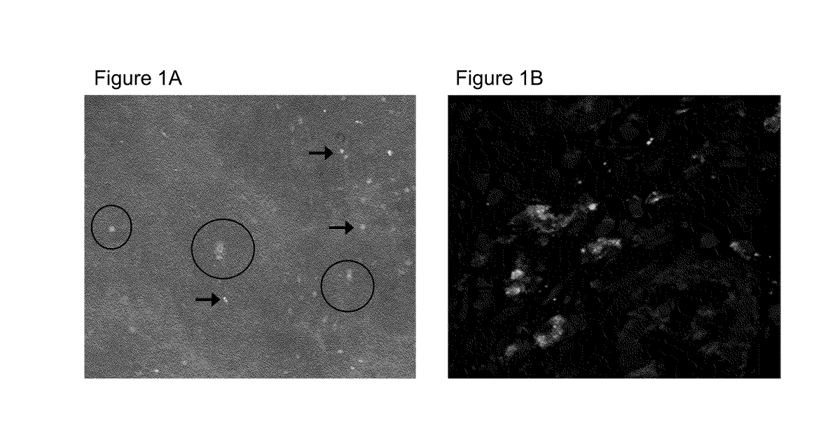

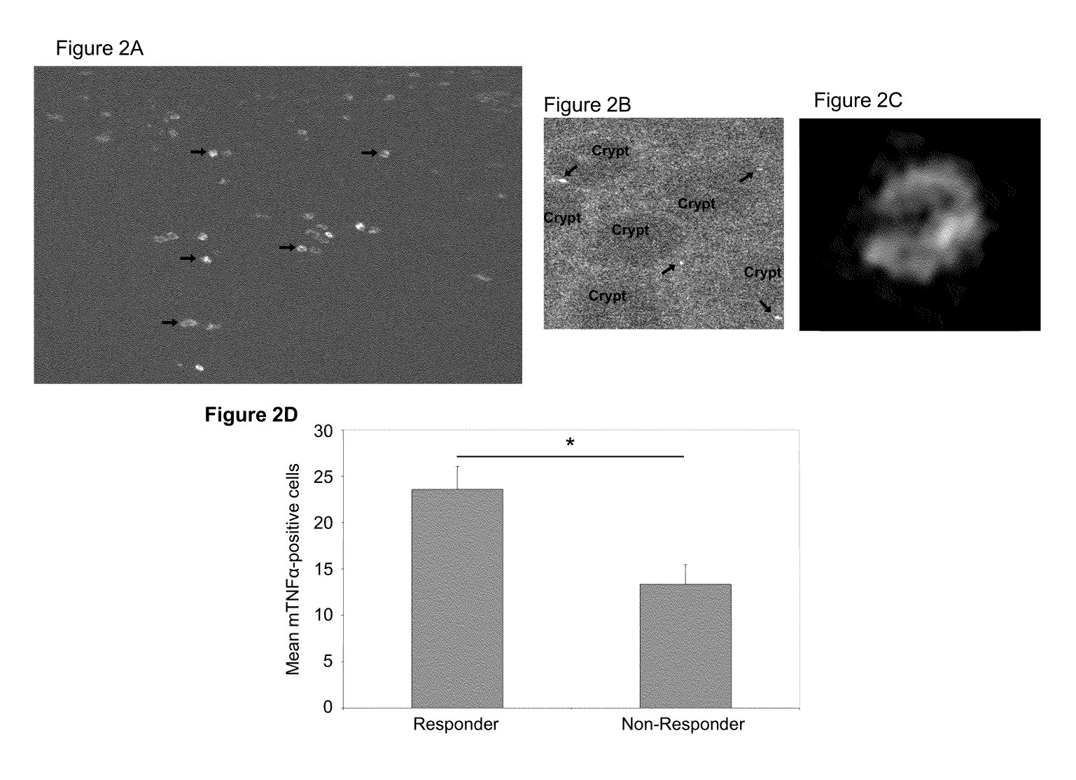

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. In one embodiment, the LCVR further has a CDR2 domain comprising the amino acid sequence of SEQ ID NO: 5 (i.e., the adalimumab VL CDR2) and the HCVR further has a CDR2 domain comprising the amino acid sequence of SEQ ID NO: 6 (i.e., the adalimumab VH CDR2). In one embodiment, the LCVR further has CDR1 domain comprising the amino acid sequence of SEQ ID NO: 7 (i.e., the adalimumab VL CDR1) and the HCVR has a CDR1 domain comprising the amino acid sequence of SEQ ID NO: 8 (i.e., the adalimumab VH CDR1). The framework regions for VL preferably are from the V_{κ}I human germline family, more preferably from the A20 human germline Vk gene and most preferably from the adalimumab VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the adalimumab VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the adalimumab VL) and a heavy chain variable region (HCVR) comprising the amino acid sequence of SEQ ID NO: 2 (i.e., the adalimumab VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing adalimumab-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

In another embodiment, the antibody or antigen-binding portion thereof, contains a light chain variable region (LCVR) comprising the amino acid sequence of SEQ ID NO: 9 and a heavy chain variable region (HCVR) comprising the amino acid sequence of SEQ ID NO: 10.

The TNFα antibody used in the methods and compositions of the invention may be modified for improved treatment of an IBD, e.g., Crohn's disease. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl-ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat IBD by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by cross-linking two or more antibodies (of the same type or of different types, e.g., to create bispecific antibodies). Suitable cross-linkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express an anti-TNFα antibody, such as adalimumab (D2E7) or an adalimumab (D2E7)-related antibody (e.g., an antibody have at least 95% identity in sequence to the amino acid sequence set forth in SEQ ID NOs: 1 and/or 2), DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of adalimumab, a member of the V_{H}3 family of human germline VH genes is amplified by standard PCR. Most preferably, the DP-31 VH germline sequence is amplified. To obtain a DNA fragment encoding the light chain variable region of adalimumab, or an adalimumab-related antibody, a member of the V_{κ}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the adalimumab, or an adalimumab-related amino acid sequences disclosed herein. The amino acid sequences encoded by the germline VH and VL DNA sequences are first compared to the adalimumab, or an adalimumab-related VH and VL amino acid sequences to identify amino acid residues in the adalimumab, or an adalimumab-related sequence that differ from germline. Then, the appropriate nucleotides of the germline DNA sequences are mutated such that the mutated germline sequence encodes the adalimumab, or an adalimumab-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding the anti-TNFα antibody, e.g., adalimumab, VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃ (SEQ ID NO: 38) such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the adalimumab, or an adalimumab-related light or heavy chain sequences, the expression vector may already carry antibody constant region sequences. For example, one approach to converting the adalimumab, or an adalimumab-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr− CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the adalimumab light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding adalimumab-related antibodies, or portions thereof (e.g., a CDR domain, such as a CDR3 domain), can be derived from the nucleotide sequences encoding the adalimumab LCVR and HCVR using the genetic code and standard molecular biology techniques.

Recombinant human antibodies of the invention in addition to adalimumab or an antigen binding portion thereof, or adalimumab-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J. 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat IBD.

### IV. KITS OF INVENTION

The invention also provides kits for assessing a subject's responsiveness to a TNFα inhibitor for the treatment of an IBD, e.g., Crohn's disease, a well as kits for treating a subject having an IBD, e.g., Crohn's disease. These kits include means (e.g., labelled anti-TNFα antibody) for determining the mTNFα expression (or presence or absence) in the intestinal mucosa of a subject and instructions for use of the kit.

One aspect of the invention includes a kit for determining if a TNFα inhibitor, e.g., a human anti-TNFα antibody, or antigen-binding portion thereof, will be effective for the treatment of a subject having inflammatory bowel disease (IBD), e.g., Crohn's disease or ulcerative colitis. To determine if the TNFα inhibitor will be effecting, the kit may include a means for determining the level of expression of TNFα in the cells of the intestinal mucosa of the subject having IBD, and instructions for recommended treatment for the subject based on the level of expression of TNFα in the cells of the intestinal mucosa of the subject having IBD. Instructions for recommended treatment will depend on the level of TNFα in the intestinal mucosa of the subject. For example, a higher level of expression of TNFα in the cells of the intestinal mucosa of the subject as compared to a control level of expression of TNFα from a nonresponder indicates that the TNFα inhibitor will be effective for the treatment of the subject having IBD, whereas an equivalent or lower level would indicate that the subject will not be responsive. Alternatively, a lower level of expression of TNFα in the cells of the intestinal mucosa of the subject as compared to a control level of expression of TNFα from a responder indicates that the TNFα inhibitor will not be effective for the treatment of the subject having IBD, whereas an equivalent or higher level of TNFα would indicate that the subject will be responsive to said treatment. In one embodiment, the TNFα level which is determined is mTNFα.

In one embodiment, the means for determining the level of expression of TNFα in the cells of the intestinal mucosa of the subject having IBD comprises a detectably labeled anti-TNFα antibody, or antigen-binding portion thereof. The anti-TNFα antibody, or antigen-binding portion thereof, may be labeled, for example, with fluorescein isothiocyanate (FITC). For example, the detectably labeled anti-TNFα antibody may be detectably labeled (e.g., FITC) adalimumab, or an antigen-binding portion thereof.

In addition to the means of determining the level of TNFα, it is contemplated that, in one embodiment, kit further comprised a pharmaceutical composition comprising a TNFα inhibitor for treatment of the subject having IBD. Examples of TNFα are provided above, and include, but are not limited to, anti-TNFα antibodies.

Thus, kits of the invention can be used to determine if a subject with IBD, e.g., Crohn's disease, will be effectively responsive to a TNFα inhibitor. These kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for a protein or a biomarker (mTNFα) gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, e.g., avidin or streptavidin, bound to a reporter molecule, such as an enzymatic, florescent, or radioisotope label.

Such a kit will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific application, and may also indicate directions for either in vivo or in vitro use, such as those described above.

Another aspect is a kit comprising a container, a label on the container, and a composition contained within the container, wherein the composition includes a primary antibody (e.g., adalimumab) that binds to a protein (i.e., mTNFα), and the label on the container indicates that the composition can be used to evaluate the presence of such proteins in a sample, and wherein the kit includes instructions for using the antibody for evaluating the presence of mTNFα in a particular sample type. The kit can further comprise a set of instructions and materials for preparing a sample and applying antibody to the sample. The kit may include both a primary and secondary antibody, wherein the secondary antibody is conjugated to a label, e.g., an enzymatic label.

Other optional components of the kit include one or more buffers (e.g., block buffer, wash buffer, substrate buffer, etc.), other reagents such as substrate (e.g., chromogen) that is chemically altered by an enzymatic label, epitope retrieval solution, control samples (positive and/or negative controls), control slide(s), etc. Kits can also include instructions for interpreting the results obtained using the kit.

In further specific embodiments, for antibody-based kits, the kit can comprise, for example: (1) a first antibody (e.g., attached to a solid support) that binds to a biomarker protein (e.g., mTFNa); and, optionally, (2) a second, different antibody that binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein-stabilizing agent. The kit can further comprise components necessary for detecting the detectable label (e.g., an enzyme or a substrate). The kit can also contain a control sample or a series of control samples that can be assayed and compared to the test sample. Each component of the kit can be enclosed within an individual container, and all of the various containers can be included within a single package, along with instructions for interpreting the results of the assays performed using the kit.

Generally, such information aids patients and physicians in using the enclosed pharmaceutical compositions and dosage forms effectively and safely. For example, the following information regarding the antagonist may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, and patent information.

In a specific embodiment of the invention, an article of manufacture is provided comprising, packaged together, a pharmaceutical composition comprising a TNFα inhibitor and a pharmaceutically acceptable carrier and a label stating that the inhibitor or pharmaceutical composition is indicated for treating patients with an IBD, e.g., Crohn's disease, from which a sample has been obtained showing the increased presence of mTNFa within the intestinal mucosa.

The kits of the invention may optionally comprise additional components useful for performing the methods of the invention. By way of example, the kits may comprise means for obtaining a biological sample from a subject, a control sample, e.g., a sample from a subject, one or more sample compartments, an instructional material which describes performance of a method of the invention and specific controls/standards.

The instructions can be, for example, printed instructions for performing the assay for evaluating the results.

The means for isolating a biological sample from a subject can comprise one or more reagents that can be used to obtain tissue, e.g., intestinal mucosa, from a subject.

Preferably, the kit is designed for use with a human subject.

The contents of all references, patents and published patent applications cited throughout this application are incorporated herein by reference

This invention is further illustrated by the following example, which should not be construed as limiting.

### EXAMPLES

Anti-TNFα antibodies have proven clinical efficacy in the treatment of inflammatory bowel disease, such as Crohn's disease (CD), but only a subgroup of patients often responds to this therapy. A method to predict the therapeutic response is much needed. Current data indicate that anti-TNFα agents mediate their effects via membrane TNFα (mTNFa) in CD. The following examples describe a study that investigated mucosal mTNFα expression and whether it could be used as a predictor of a subject's response to an anti-TNFα therapy.

The study below describes the prediction of anti-TNFα antibody responses in CD by endoscopic molecular imaging in vivo and ex vivo, and includes results from a clinical phase 1 study. The study shows that in vivo and ex vivo molecular imaging using fluorescent anti-TNFα antibodies predicts response to biological therapy in patients having CD.

### Example 1

### Prediction of Responsiveness to Anti-TNFα Antibody For Treatment of Inflammatory Bowel Disease (IBD)

Biological therapy with antibodies against TNFα has revolutionised treatment of inflammatory bowel diseases, such as Crohn's disease (CD). Sometimes, however, only a subgroup of patients responds to anti-TNFα therapy. As anti-TNFα antibodies suppress immune responses in CD by binding to membrane TNFα (mTNFα) expressing effector cells, the following study examines whether in vivo and ex vivo detection of such cells might be used for prediction of therapeutic efficacy. In order to test the predictive nature of mTNFα, a GMP (Good Manufacturing Practice)-conform, fluorescent anti-TNFα antibody was developed for in vivo molecular imaging. Topical administration of the anti-TNFα antibody in 25 CD patients led to detection of mTNFα positive immune cells in the gut during confocal laser endoscopy. Patients with high amounts of mTNFα positive cells showed significantly higher response rates at week 12 (92%) upon subsequent anti-TNFα therapy as compared to patients with low amounts of mTNFα positive cells (15%). This clinical response in the former patients was sustained over a follow-up period of one year. These data indicate for the first time that molecular imaging with fluorescent antibodies can predict therapeutic responses to biological treatment and open new avenues for personalized medicine by using fluorescent antibodies in CD and other autoimmune and chronic inflammatory diseases.

### Materials and Methods

One of the goals of the following clinical phase 1 study was to visualize mucosal mTNFα expression in humans using confocal laser endomicroscopy (CLE) with topical application of fluorescin isothiocyanate-labeled adalimumab. This novel in vivo diagnostic modality was used to predict clinical response to subsequent adalimumab therapy in CD patients. Prospectively, 15 CD patients with an indication for anti-TNFα treatment were included in this study. Fluorescin isothiocyanate-labeled adalimumab was topically applied via a spray catheter onto the inflamed mucosa of CD patients during colonoscopy prior to anti-TNFα therapy. Fluorescein expression on a cellular level, indicating intestinal mTNFα positive cells, was identified and quantified via CLE. CD patients were then treated with adalimumab and changes to the CDAI score were correlated to the amount of mTNFα positive cells in the mucosa. Response to treatment was defined as a decrease in the CDAI over 100 points from baseline after 12 weeks.

Details of the materials and methods used in the study are provided in more detail below:

Labelling of Adalimumab with Fluorescein Isothiocyanate

The labeled antibody was manufactured in the GMP unit of the Department of Pharmacy at the Erlangen University Hospital according to GMP-requirements. Fluorescein isothiocyanate was covalently conjugated to the fully human IgG1 monoclonal anti-TNFα antibody adalimumab (Abbott Laboratories) using specific labeling reagents (Thermo Fisher Scientific). The concentration of adalimumab was adjusted to 2 mg/ml with 50 mM borate buffer (pH 8.5). The diluted protein was added to a vial containing fluorescein isothiocyanate (96 nmol). The sample was mixed and kept for 1 hour at room temperature protected from light. Dye Removal Columns were used to remove free fluorescein isothiocyanate, as described by the manufacturer (Thermo Scientific). The absorbance of the solution containing the fluorescein isothiocyanate labeled IgG was determined at 280 and 495 nm. The binding molar ratio (F/P ratio) of fluorescein isothiocyanate to IgG was calculated as follows: F/P molar ratio=2.97 A₄₉₅/(A₂₈₀−0.32 A₄₉₅). The study product contained 1.07 μg/μl labeled adalimumab.

### SDS-PAGE Electrophoresis of Fluorescent Adalimumab.

Labeled adalimumab and purified human IgG (Innovative Research) were analyzed by the SDS-PAGE Phast System (Amersham Biosciences) or SDS-PAGE Laemmli system. A sample containing 1 μg of fluorescent adalimumab was boiled for 5 minutes in sample buffer containing mercaptoethanol. The whole sample was applied to a 12% acrylamide separating gel (Bio-Rad Mini-PROTEAN Tetra cell). The gel was exposed to UV light for the detection of fluorescence (Bio-Rad Molecular Imager XR+System) and to exclude the presence of unbound fluorescein isothiocyanate in the fluorescent adalimumab working solution. Thereafter, the gel was stained by Coomassie.

### Patient Population

The molecular imaging studies in vivo were performed as a prospective, monocentric, open-label, one-arm clinical study. The trial was registered at clinicaltrials.gov (Study NCT01275508).

Twenty-five patients with histologically and clinically confirmed CD who had active disease as defined by the CDAI score of >150 points were prospectively included in this study. Furthermore, all patients were required to have the clinical indication for an anti-TNFα therapy due to their clinical course of the disease (e.g. steroid and immunosuppressive refractory disease) (Hueber et al. (2010) Sci Translational Med 2:52-72). Concurrent therapies for CD, including 5-aminosalicylates, prednisone (≦30 mg/day), azathioprine and antibiotics were permitted at stable dosages. Female patients with childbearing potential were required to use a highly effective form of birth control (failure rate of <1% per year).

Patients were excluded if they had impaired blood clotting, underwent extensive bowel resection (>100 cm), had a short bowel syndrome, were receiving total parenteral nutrition, were pregnant or breast feeding or had received enema therapy within one month prior to inclusion in the study. Patients with an anti-TNFα therapy within the last 12 months were also excluded. Furthermore, participation in any other clinical trial or administration of any investigational drug within the last four months prior to the screening visit was not allowed. Other contraindications included moderate to severe heart failure, active tuberculosis or acute infections.

### Ex Vivo Molecular Imaging

A hand held rigid confocal probe (FIVE1, Optiscan) was used for ex vivo studies (Foersch et al. Gut 59, 1046-1055 (2010)). The blue laser light incorporated an excitation spectrum of 488/505-585 nm, obtaining optical sections of 475×475 μm. The lateral resolution was 0.7 μm and the optical slice thickness was 7 μm. The depth of this device could be adjusted until 250 μm.

Ex vivo molecular imaging was performed using ex vivo surgical gut samples of CD patients who underwent surgery (n=5). Tissue samples were repeatedly rinsed with phosphate buffered saline (PBS). The samples were then incubated with the study product (20 μg labeled adalimumab/500 μl PBS) for 1-10 minutes. After washing the tissue with PBS to remove unbound antibody, specimens were scanned by confocal laser endomicroscopy (CLE).

Part of the stained intestinal tissue was immediately frozen and cut into histological sections for further immunohistochemical analysis. These slides were counter-stained with an anti-fade medium containing DAPI (Vector Laboratories) and imaged using a SP-5 confocal microscope with a 63×/1.3 NA objective (Leica Microsystems).

### Histological Evaluation

Paraffin embedded sections of formalin-fixed biopsies from the imaged mucosal areas were stained by H&E and analyzed blindly by pathologists at the Klinikum of Bayreuth and the University of Erlangen (n=25). Histological scoring for the severity of acute inflammation was based on the infiltration rate of neutrophilic granulocytes in the diseased tissue. The histological score ranged between 0 (no acute inflammation) and 3 (massive acute inflammation).

### Immunohistochemistry and Confocal Microscopy

Immunohistochemistry was performed on paraffin-embedded sections of the intestinal biopsies taken during the endoscopic examination. After fixation with 4% PFA and conventional staining procedure, slides were incubated overnight with labelled adalimumab. Further staining was performed with fluorescein isothiocyanate-labeled immunoglobulin (Ig) G1 (BD PharMingen). Sections were counterstained with mounting medium (Vector Laboratories) and analyzed with an immunofluorescence (Olympus) or a confocal microscope (Leica Microsystems). Cells in 3 high-power fields were counted per slide in all patients.

### Study Design

Twenty-five patients with active CD and indication for anti-TNFα treatment were included in this study. Patients were assessed at weeks −1 (Visit 1), 0 (Visit 2), 1 (Visit 3), 5 (Visit 4) and 13 (Visit 5) with additional telephone interviews at days 1, 14 and 21. The screening of the patients was performed during Visit 1. At Visit 2, the molecular imaging in vivo was performed. At Visit 3, subcutaneous adalimumab therapy was initiated (160 mg at visit 3 and 80 mg two weeks thereafter). This was followed by application of adalimumab 40 mg every other week. The CDAI score was assessed during the visits 1, 3, 4 and 5. A response was defined as a reduction of the CDAI score ≧100 points at visit 5 as compared to visit 3. Crohn's disease patients with a high mTNFα expression were followed up for 52 weeks after the induction of the adalimumab therapy. The CDAI score was assessed in these patients during the visit. Adverse events were recorded throughout the study. Normal CRP levels were defined as CRP values <0.5 mg/l. The demographics of the patient population is described in Table 1.

TABLE 1
Baseline demographics and clinical characteristics of the CD
patients enrolled in the molecular imaging study.

| | Low mTNFα | |
|---|---|---|
| | (13 of 25 | High mTNFα |
| Characteristic | patients) | (12 of 25 patients) |
| Female patients, n (%) | 4 (30.8) | 5 (41.7) |
| Age (y), mean (SD) | 39.1 (13.1) | 44.2 (16.3) |
| Body wt (kg), mean (SD) | 71.9 (11.5) | 75.3 (10.6) |
| Disease duration (y), mean (SD) | 13.1 (5.0) | 9.5 (10.6) |
| Involved intestinal area, n (%) | | |
| Colonic | 9 (69.2) | 6 (50.0) |
| Ileal | 9 (69.2) | 8 (66.7) |
| Gastroduodenal | 1 (7.7) | 3 (25.0) |
| Enterocutaneous or perianal fistula, | 3 (23.1) | 0 |
| n (%) | | |
| Median CRP (mg/l) | | |
| Baseline (SD) | 18.1 (22.3) | 8.8 (16.8) |
| 1 Month (SD) | 19.1 (23.6) | 8.2 (17.5) |
| 3 Months (SD) | 19.7 (35.6) | 3.0 (3.90) |
| Previous TNF antagonist exposure, | 1 (7.7) | 1 (8.3) |
| n (%) | | |
| Concomitant medication, n (%) | | |
| 5-Aminosalicylates | 2 (15.4) | 5 (41.7) |
| Corticosteroids | 7 (53.9) | 6 (50.0) |
| Azathioprine | 4 (30.8) | 5 (41.7) |
| Antibiotics | 1 (7.7) | 0 |
| Current smoker, n (%) | 0 (0.0) | 4 (33.3) |

### In Vivo Molecular Imaging

In vivo molecular imaging was performed in CD patients during routine colonoscopy prior to the initiation of adalimumab therapy. All patients were routinely prepared for colonoscopy using Moviprep (Norgine) for adequate bowel cleansing. Endoscopic examination of the ileum and/or the colon was performed using a conventional white light video endoscope in which a confocal fluorescence microscope is integrated into the distal tip (Pentax Endomicroscopy System) (Neumann et al. Gastroenterology 139, 388-392, 392 e381-382 (2010)). The endoscope-integrated confocal microscope (iCLE) collected images at a scan rate of 1 frame per second yielding a resolution of 1024×1024 pixels (1 megapixel) with a dynamically adjustable depth of scanning ranging from 0 to 250 μm. This system used an incident 488 nm wavelength laser and enabled the detection of fluorescence between 205 and 585 nm wavelengths. The lateral and axial resolution was 0.7 μm, enabling a confocal image view of 475×475 μm. The laser power could be adjusted between 0 and 1000 μW.

Conventional white light endoscopy was performed to select suitable intestinal areas for subsequent endomicroscopic examination. Mucosal sites with the heaviest inflammation were selected for the endomicroscopic procedure. Sites with ulcers and active bleeding were excluded from this study due to the risk of imaging artifacts. The mucosal site of interest was washed with water, which was applied through a spray catheter, to remove excess mucus. Before application of labeled adalimumab, the mucosa was endomicroscopically inspected to exclude unspecific background signals due to autofluorescence of the tissue. Next, 20 μg of labeled antibody was topically administered in a 4 ml watery dilution to the surface of the mucosa via a standard spraying catheter (Olympus). After an incubation time of one minute, excess antibody was removed by gently rinsing the mucosa with water. Afterwards, imaging was performed using the endomicroscopic confocal fluorescence imaging system which uses a laser light with a wavelength of 488 nm that was emitted via the confocal optics to excite the fluorescent dye. The labelled moiety of the adalimumab antibody, which was bound to mTNFα positive lamina propria cells, reflected the emitted laser light by confocal laser endomicroscopy with a wavelength of 518 nm. The reflected light waves therefore enabled detection of fluorescent adalimumab binding on a cellular level, indicating mTNFα positive cells in the lamina propria. Digital images of the area were stored for documentation and later analysis. We collected fluorescence images in vivo at 1 frame per second up to depths of 50 μm beneath the mucosal surface at a resolution of 1 megapixel. This staining procedure was done in the same mucosal area four times altogether. Each CD patient topically received 80 μg adalimumab, 183 ng fluorescein and 32 ng isothiocyanate altogether. Mean imaging time was approximately 15 minutes per patient. The signal-to-background ratio (SBR) (ratio between the mean pixel value of mTNFα positive cells and the pixel values in a homogenous block of pixels in the tissue) and the signal-to-noise ratio (SNR) (ratio between the mean pixel values of mTNFα positive cells against average signal in the imaging field outside the specimen or instrument noise) were mathematically calculated in 50 representative confocal images.

Afterwards biopsies were taken with a standard endoscopic forceps instrument from the imaged mucosal area and submitted for histopathological evaluation. In addition, samples for ex vivo staining with labelled adalimumab were taken adjacent to the imaging areas, as specified below. At the end of the examination, the endoscope was re-advanced to the inspected mucosal area for a macroscopic assessment regarding signs of local intolerance to the study product.

### Statistical Analysis

Tests for significance of differences were made by Student t tests using Excel (Microsoft Corp, Redmond, Wash.). Differences with a P value of ≦0.05 were considered significant.

### Results

### Development of a GMP Conform, Fluorescent Anti-TNFα Antibody for Molecular Imaging

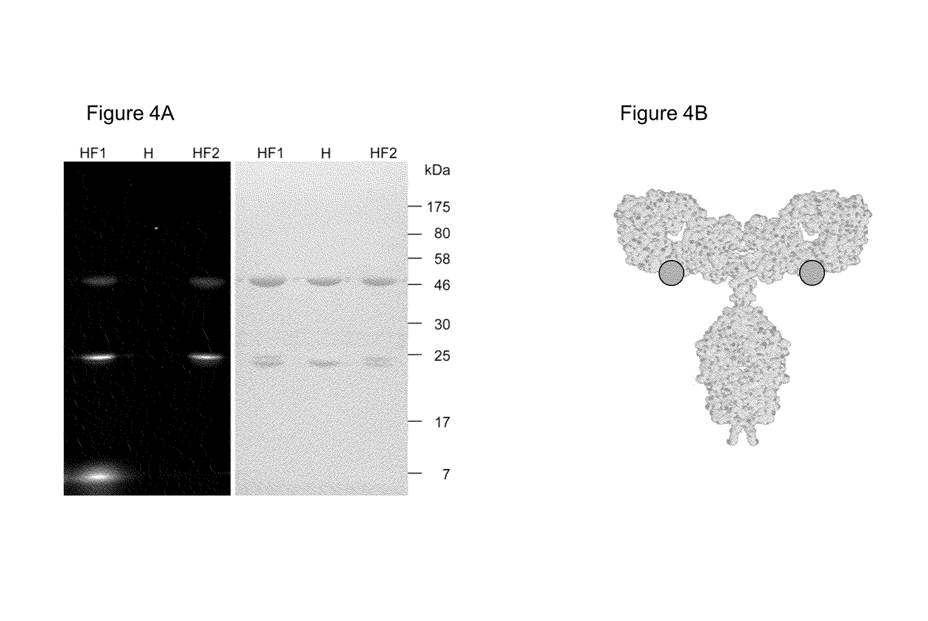

The following study determined whether molecular imaging of the expression of mTNFα in the gut immune cells could be used to predict a clinical response to anti-TNFα therapy. In order to permit visualization of the binding of the anti-TNFα inhibitor to mTNFα through confocal laser endomicroscopy, adalimumab was labelled with fluorescein isothiocyanate under GMP conditions for in vivo use. Adalimumab was labeled with fluorescein isothiocyanate under GMP conditions for in vivo use in order to permit visualization through confocal laser endomicroscopy. On average one adalimumab molecule was labelled with 2.1 fluorescein molecules at 25 degrees C. Subsequently labelled antibodies were analysed by gel electrophoresis and Coomassie staining. Detailed analysis demonstrated that there was no free unbound fluorescein isothiocynate after the labelling procedure (as shown in FIG. 4A).

FIG. 4 provides an analysis of fluorescent adalimumab. FIG. 4A provides an SDS PAGE gel showing electrophoresis of fluorescein isothiocyanate-adalimumab, which was labeled with a 14-fold excess of fluorescein isothiocyanate (HF1) and adalimumab (H) and fluorescein isothiocyanate-adalimumab after removing the excess fluorescent dye with a dye removal column according to the study protocol (HF2). The left panel of FIG. 4A depicts the fluorescence when the gel was exposed to UV light. The right panel of FIG. 4A shows the gel after Coomassie staining. All lanes contained 1 μg of the protein. (b) Hypothetical model of fluorescent adalimumab based on the above analysis.

Ex Vivo Molecular Imaging of mTNFα-Positive Mucosal Cells with Fluorescent Adalimumab in Intestinal Tissue of Crohn's Disease Patients

To test the specificity of the labeled antibody for mTNFα binding, surgical specimens of CD patients were shielded from light and incubated with fluorescent adalimumab for 10 minutes at room temperature. After washing with PBS confocal imaging was performed using the FIVE1 probe. Ex vivo confocal imaging revealed a specific fluorescence signal that allowed identification of mTNFα expressing mucosal cells in the inflamed tissue of CD patients (FIG. 1A). Subsequent analysis of sections from these specimens by bench top fluorescence microscopy confirmed the fluorescence signal of mTNFa-positive lamina propria mononuclear cells within the intestinal tissue after nuclear counterstaining (FIG. 1B).

In Vivo and Ex Vivo Molecular Imaging of mTNFα Positive Immune Cells in the Gut of CD Patients

The labeled antibody was then used for in vivo and ex vivo molecular imaging of the mucosa in patients with CD. As labeled adalimumab had not been used in human subjects before, approval by the federal authorities was obtained. Subsequently, endoscopic examination with the fluorescent antibody was performed in 25 patients with active CD (CDAI>150 points) prior to adalimumab therapy. Accordingly, labeled adalimumab was topically applied via a spray catheter onto the most inflamed region of the bowel during colonoscopy prior to clinical anti-TNFα therapy. Fluorescence of intestinal mTNFα positive cells was detected and quantified via confocal laser endomicroscopy.

In vivo imaging of inflamed areas of the intestinal mucosa of CD patients showed a specific fluorescence signal of mTNFα positive cells after topical application of labeled adalimumab (see FIG. 2A which provides a representative image). These specific fluorescence signals were markedly greater than background autofluorescence. The mean signal to background ratio in CD patients was 9.74+/−2.4 (s.d.), whereas the signal to noise ratio was 10.96+/−1.9 (s.d.). Contrast enhanced imaging showed that positive cells were localized outside of the crypt in the lamina propira (see FIG. 2B). Detailed inspection by high magnification revealed a membranous fluorescence pattern of mTNFα positive cells upon topical administration of fluorescent adalimumab in vivo (FIG. 2C) that was comparable to confocal microscopic images of mTNFα expressing cells from biopsies in the same patients. Biopsy cryosections were made and analyzed by immunohistochemistry upon staining with adalimumab and counterstaining with DAPI. Administration of labeled adalimumab was well tolerated in all patients and no adverse events were noted.

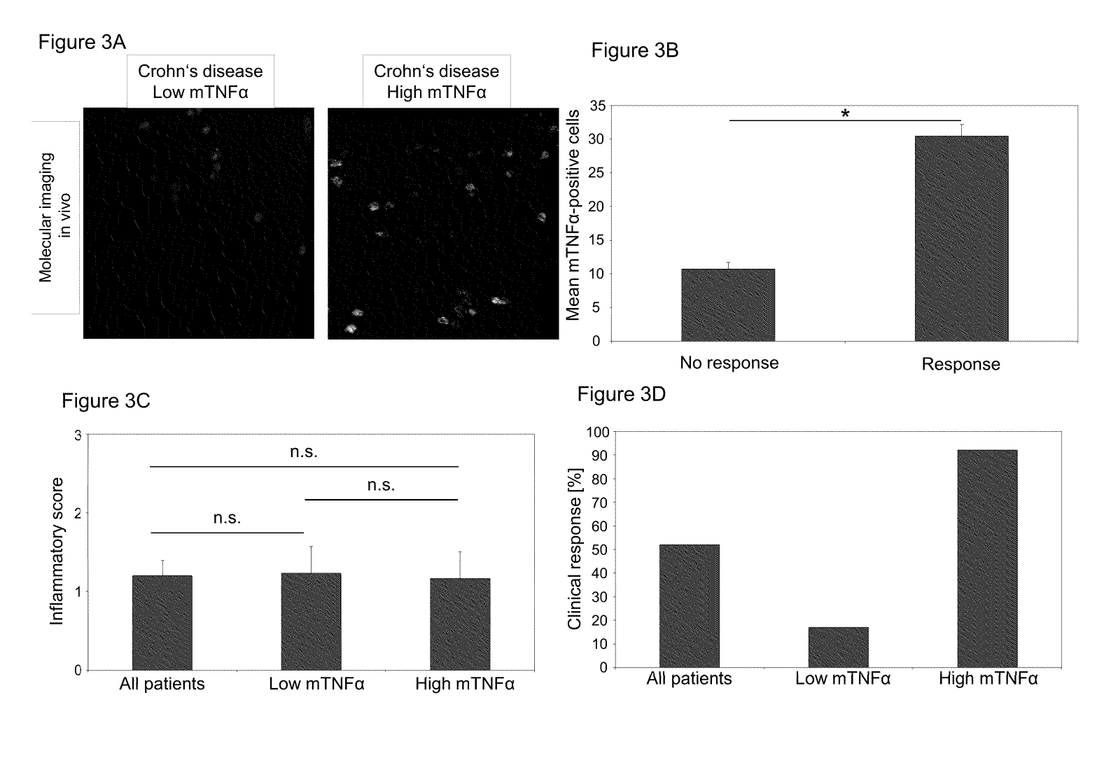

Although the inflamed mucosal areas in CD examined during the molecular imaging procedure with fluorescent adalimumab had similar macroscopic signs of inflammation during conventional endoscopy, there were nevertheless marked inter-individual differences regarding the number of mucosal mTNFα positive cells (see FIGS. 3A and 3B). High-resolution endoscopic images of the inflamed mucosa (sigmoid colon) of CD patients with low or high numbers of mTNFα positive immune cells were examined. Marked mucosal inflammation with edema, swelling of the mucosa and hyperemia was visible. In spite of similar levels of mucosal inflammation, molecular in vivo imaging with fluorescent adalimumab revealed low (FIG. 3A, left panel) and high (FIG. 3A, right panel) numbers of mTNFα-expressing immune cells in the above patients.

Following in vivo molecular imaging, patients received adalimumab therapy over a period of 12 weeks followed by assessment of clinical responses to therapy. Response to adalimumab therapy was defined as a drop of more than 100 points in the CDAI score 12 weeks after in vivo molecular imaging and initiation of therapy. Quantification of the median of mTNFα positive cells obtained by in vivo molecular imaging in patients with or without response to adalimumab therapy is shown in FIG. 3B. There were a significantly lower number of mTNFa+cells in patients without response to subsequent adalimumab therapy as compared to patients with response to anti-TNF therapy (mean values±s.e.m.; *p=0.00003).

In spite of similar histological scores of inflammation (FIG. 3C) quantitative analysis of the in vivo images (based on analysis of 8 confocal images measuring 475 μm×475 μm per patient) revealed one group of CD patients with high numbers of mTNFα positive mucosal immune cells (>20 cells/confocal image), while the other group had low amounts of mTNFα expressing cells (<20 cells/confocal image) in the lamina propria (FIG. 3A, 3B).

### Clinical Outcome Analysis

Following in vivo imaging with labeled adalimumab during confocal laser endoscopy, all 25 CD patients with active disease were treated with adalimumab and the clinical response to anti-TNFα treatment was evaluated. The study outline can be described as follows: Patients with active CD and indication for anti-TNFα treatment were screened at week −1 (day−7). Molecular in vivo imaging was performed at day 0. The baseline CDAI score was assessed at day 7, when adalimumab treatment was also initiated with a 160 mg dose of the adalimumab antibody given subcutaneously. Treatment was continued with 80 mg adalimumab s.c. at day 21, and 40 mg adalimumab were given every other week until day 91. The CDAI score was assessed at days 35 and 91. Additional telephone interviews were conducted at days 1, 14 and 21 to record potential adverse advents.

The clinical analysis showed that 52% (13) of the CD patients had a clinical response (as defined as a decrease of the CDAI score ≧100 points) after 12 weeks of adalimumab treatment. The mean number of in vivo detected mTNFα positive cells per patient was then correlated to the clinical outcome of adalimumab therapy. It was shown that the mean number of mTNFα positive cells/confocal image was 11±1 in CD patients without subsequent clinical response to adalimumab treatment, while a mean number of 30±1.7 mTNFα expressing cells per confocal image was detected in patients with clinical response (see FIG. 3B).

To confirm these in vivo molecular imaging results, histological gut sections from the mucosal area adjacent to the site where molecular imaging was performed were stained ex vivo with labeled adalimumab. CD patients with low and high numbers of mTNFα expressing mucosal immune cells could be differentiated by ex vivo staining. Quantitative analysis of ex vivo staining demonstrated that patients with clinical response to adalimumab therapy after 12 weeks had a significantly higher number of mTNFα expressing cells (mean number of 24 mTNFα expressing cells/high power field) than patients without clinical response (mean number of 13 mTNFα expressing cells/high power field). FIG. 2D depicts quantitative analysis of ex vivo staining demonstrating that CD patients with clinical response to adalimumab therapy after 12 weeks had a significantly higher number of mTNFα expressing immune cells than patients lacking clinical response to adalimumab therapy. Ex vivo images were magnified by a SP-5 confocal microscope with a 63×/1.3 NA objective (Leica Microsystems

Due to the statistical difference regarding the in vivo mTNFα expression between patients with and without clinical benefit in adalimumab therapy, the sensitivity and specificity for the prediction of clinical response to adalimumab treatment based on a discriminative factor of 20 mTNFa-positive cells/confocal image was assessed. Accordingly, CD patients were stratified into high mTNFα (≧20 cells/confocal image) and low mTNFα (<20 cells/confocal image) groups based on the mean number of mTNFα expressing cells per confocal high power field (475 μm×475 μm). These groups demonstrated neither a significant difference in inflammatory activity in the colon (FIG. 3C) nor in systemic CRP levels (see Table 1). However, it was found that CD patients with high numbers of mTNF expressing cells per confocal image (high mTNFα: ≧20 cells/confocal image) demonstrate a markedly higher probability of clinical response to subsequent adalimumab therapy than patients with low numbers of mTNFα positive cells (low mTNFα: <20 cells/confocal image) (92% versus 15%; FIG. 3D). The sensitivity, specificity and accuracy for the prediction of therapeutic responses were 92%, 85% and 88%, respectively. Positive and negative predictive values were 85% and 92% (Table 2).

TABLE 2

| | Prediction of |
|---|---|
| Characteristic | clinical response (%) |
| Sensitivity | 92 |
| Specificity | 85 |
| Accuracy | 88 |
| Negative predictive value | 92 |
| Positive predictive value | 85 |

Table 2 describes the sensitivity, specificity and accuracy for the prediction of clinical response to adalimumab treatment based on a discriminative threshold of ≧20 mTNFα positive cells (mean in confocal laser endomicroscopic images). Positive and negative predictive values regarding clinical response based on ≧20 mTNFα positive cells per confocal image.

Further analysis 4 and 12 weeks after adalimumab therapy revealed that patients in the high mTNFα group exhibited a statistically significant reduction of their CDAI level, while patients in the low mTNFα group had no significant reduction in their CDAI score. The mean CDAI score (±s.e.m.) in the former group was 253±29 prior to adalimumab treatment, while mean values of 117±34 and 93±29 were noted at 4 and 12 weeks after therapy, respectively. In contrast, patients in the latter group showed no significant reduction in CDAI scores after therapy: there was a mean CDAI score of 295±31 prior to adalimumab treatment, which changed to 238±35 after 4 weeks and to 249±52 after 12 weeks of adalimumab treatment.

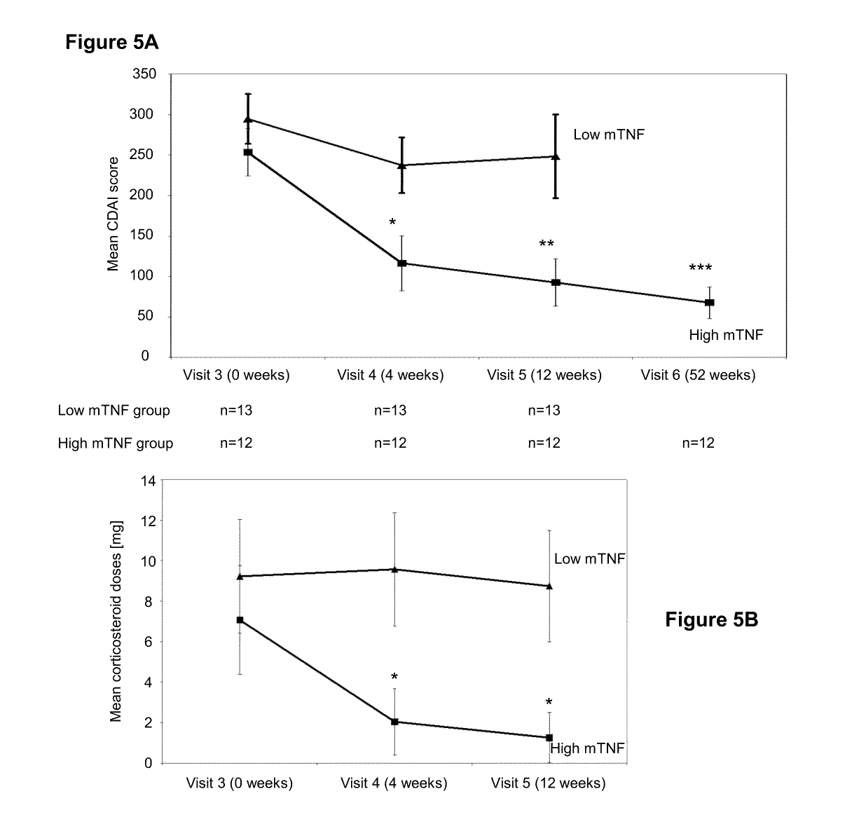

In addition, it was shown that patients with high mTNFα expression had a significant reduction of the mean corticosteroid use in the course of adalimumab therapy. Mean (±s.e.m.) values changed significantly from 7.1±2.7 mg/d before therapy to 2.0±1.7 mg/d and 1.25±1.2 mg/d after 4 and 12 weeks of adalimumab treatment (p=0.04), respectively. In contrast, patients with low amounts of mTNFα expressing cells showed no significant changes: 9.2±2.8 mg/d before treatment and 9.6±2.8 mg/d and 8.75±2.8 mg/d after 4 and 12 weeks of adalimumab treatment (FIGS. 5A and 5B), respectively.

Following the 12 week therapy, all responders in the high mTNFα group received extended therapy with adalimumab over a period of 12 months, while non-responders in the low mTNFα group were switched to other therapeutic regimens. The clinical follow-up of the responders in the Crohn's disease group with high mTNFα expression over one year after the induction of the adalimumab therapy demonstrated a sustained highly significant reduction of their CDAI level with a mean value of 68±20 at week 52 (see FIG. 5A). In contrast to the low mTNFα group where 4 patients had to undergo surgery (3 patients due to stenosis, 1 patient due to conglomerate tumor), none of the patients in the high mTNFα group had to undergo surgery within 12 months of adalimumab therapy underlining the different responses of the two groups to clinical anti-TNFα therapy.

### Discussion

The above results demonstrate that fluorescent anti-TNFα antibodies and confocal laser endomicroscopy can be used for in vivo molecular imaging of mucosal immune cells in CD patients during a colonoscopy. Quantitative assessment of the number of immune cells expressing mTNFα was achieved and can be used to predict clinical response to subsequent treatment with the anti-TNFα antibody adalimumab in CD. This is the first report on the use of GMP conform, fluorescent antibodies for in vivo imaging in humans. These findings suggest that fluorescent antibodies have a high potential for in vivo imaging in humans with broad applications in clinical medicine.

Confocal laser endomicroscopy has recently emerged as a novel technique for performing real time in vivo imaging of the mucosa at cellular and subcellular levels (Neumann et al. Gastroenterology 139, 388-392, 392 e381-382 (2010) and Kiesslich et al. Nat Clin Pract Oncol 4, 480-490 (2007)). Additional studies revealed that this technique may be utilized for in molecular imaging procedures (Hsiung et al. Nat Med 14, 454-458 (2008)). However, in vivo molecular imaging using endomicroscopy in humans was restricted to labelled peptides with relatively low binding affinity to target structures. Here, the above study took advantage of an anti-TNFα monoclonal antibody that exhibits a high affinity to human mTNFα, and used this antibody upon specific fluorescence labelling under GMP criteria for in vivo imaging during colonoscopy in CD. Imaging was performed upon topical administration of fluorescent antibody to the most inflamed part of the gut mucosa in active CD to identify mTNFα expressing cells, as it was suggested that this area would adequately reflect the highest inflammatory burden for subsequent adalimumab therapy. As the bather function of intestinal epithelial cells is markedly impaired in active CD (Salim and Soderholm Inflamm Bowel Dis 17, 362-381 (2011)) and mTNFα is expressed on the outer membrane of mucosal immune cells (Atreya (2011) ibid.), topical administration offered the advantage of rapid access to the region of interest and was an ideal approach for delivery of the molecular probe in CD.

Minimal concentrations of antibody (3 orders of magnitude lower than systemic adalimumab therapy in CD) were sufficient for successful topical visualization of mTNFα positive cells in intestinal biopsies by using ex vivo confocal imaging using a hand held probe, which also minimized the potential risk of allergic reactions to fluorescent adalimumab. This concept led to the approval of topical administration of the fluorescent antibody for diagnostic clinical use in this in vivo molecular imaging study by the Paul-Ehrlich Institute as regulatory authority for antibody use in Germany. The application of this diagnostic procedure was easily implementable into clinical practice, as colonoscopies are routinely performed in CD patients before anti-TNFα therapy is initiated to exclude possible superinfections and to estimate the extent and severity of mucosal inflammation (Wilkins et al. American family physician 84, 1365-1375 (2011) and Neurath and Travis Gut (2012)).

Local administration to the intestinal mucosa was safe and no adverse events were noted indicating that topical application of unlabeled adalimumab by spray catheter during colonoscopy is an acceptable method of administration of TNFα inhibitor to a patient for the treatment of CD. Topical administration of fluorescent adalimumab allowed molecular in vivo imaging of mTNFα positive cells with high signal to noise and signal to background ratios. Similarly, recent reports on molecular imaging using topically delivered fluorescent lectins or labelled heptapeptides showed high signal to noise and signal to background ratios suggesting that local administration of fluorescent agents may result in substantially better values as compared to results obtained after systemic administration of antibody-based agents (Hsiung et al. (2008) ibid.; Bird-Lieberman et al. 18, 315-321 (2012); Medarova et al. Cancer Res 69, 1182-1189 (2009); and Kobayashi, et al. Clinical cancer research: an official journal of the American Association for Cancer Research 10, 7712-7720 (2004)). Thus, topical administration of fluorescent adalimumab to the intestinal mucosa enabled rapid visualization of mTNFα expression on a cellular level. These findings are likely related to the known high binding affinity of adalimumab to mTNFα, and the observation that fluorescent antibodies rapidly reached the mucosa where they bound to mTNFα positive immune cells. These cells have been previously characterized as lamina propria CD14+ macrophages and CD4+ T cells and are known to play a fundamental role in CD pathogenesis (Monteleone, G., et al. Current opinion in pharmacology 11, 640-645 (2011); Kamada, N., et al. J Clin Invest 118, 2269-2280 (2008); and Kamada, N., et al. J Immunol 183, 1724-1731 (2009)).

Several studies indicated that clinically effective anti-TNFα antibodies work by inducing T cell apoptosis via binding to mTNFα expressing target cells in CD (Van den Brande et al. Gut 56, 509-517 (2007); Van den Brande et al. Gastroenterology 124, 1774-1785 (2003); and Mitoma et al. Gastroenterology 128, 376-392 (2005)). It was therefore of interest to correlate the results from in vivo and ex vivo molecular mTNFα imaging with clinical data from subsequent anti-TNFα therapy using a TNFα inhibitor, i.e., adalimumab. In spite of the presence of similarly active mucosal inflammation in all patients, it was found that patients with high numbers of mTNFα positive immune cells show significantly higher response rates to adalimumab therapy as compared to patients with low numbers of mTNFα positive cells. This finding was associated with a significantly lower corticosteroid use in the former as compared to the latter patients. Similarly, histological assessment of mucosal mTNFα expression in intestinal biopsies of CD patients also showed a significant correlation with the subsequent response to adalimumab therapy. While this correlation was weaker than the correlation found in the in vivo molecular imaging study, it was possibly due to antigen alterations during tissue processing and staining. Nonetheless the ex vivo results support the same conclusion and can be used for predictive methods as well. The presence of similar levels of mucosal inflammation in all patients suggested that the difference in mTNFa-expressing cells in vivo between both groups was not the result of varying immune cell infiltration levels but might rather be explained by a divergence of mTNFα expressing cells between the patients. These findings suggest that the low clinical response rate in CD patients with low numbers of mTNFα positive immune cells is at least partially due to the absence of anti-TNFα target cells in the inflamed gut, and is thus consistent with the idea of mechanistic failure of adalimumab therapy in mTNFa-independent CD inflammation.

Molecular imaging with monoclonal antibodies in humans is currently restricted mainly due to the limitation of using fluorescently labeled antibodies in vivo. In the present study, fluorescent monoclonal antibodies were used for the first time for molecular imaging in CD patients in vivo to establish a biomarker to differentiate between unlikely and likely responders to a disease-specific therapy, i.e., an anti-TNFα inhibitor. Thus, fluorescent antibodies appear to have significant potential to serve as biomarkers for decisions on subsequent therapy with biological agents. For instance, in the field of gastrointestinal disorders, endomicroscopic imaging with fluorescent antibodies would be suitable for other autoimmune and chronic inflammatory diseases such as ulcerative colitis, where anti-TNFα agents have been successfully used in subgroups of patients. Moreover, in gastrointestinal tumours, labeled antibodies against EGFR or VEGF could be used for novel diagnostic approaches aiming at predicting subsequent therapeutic responses in cancer patients. This concept is supported by recent studies on molecular imaging in colorectal cancer identifying VEGF expressing cells using anti-VEGF antibodies and endomicroscopy in xenograft models and tumor samples ex vivo (Foersch et al. (2010) Gut 59:1046-1055). Thus, this approach might be particularly attractive for colorectal cancer, as anti-EGFR and VEGF antibodies have been shown to induce clinical responses in subgroups of patients and are used in clinical routine for therapy of this disease. Finally, given the recent success of neutralizing monoclonal anti-cytokine antibodies (e.g. anti-TNFα, anti-IL-6R, anti-IL-17A antibodies) in subgroups of patients with autoimmune and chronic inflammatory diseases such as rheumatoid arthritis and psoriasis, in vivo molecular imaging with labeled antibodies could also be used for prediction of responders to therapy in these diseases upon topical administration of labeled antibodies (e.g. epidermal or intraarticular administration). Thus, molecular imaging with fluorescent antibodies emerges as an approach for identifying responders to therapy in patients with chronic inflammatory and autoimmune disorders, as well as in cancer. The above data shows for the first time that molecular imaging with fluorescent antibodies can predict therapeutic responses to biological treatment. This approach might open new avenues for personalized medicine in CD and other inflammatory disorders.

### CONCLUSION

As anti-TNFα antibodies appear to induce immunosuppression in CD by binding to mTNFα on target cells in the mucosal immune system (Atreya, et al. (2011) ibid., and ten Hove et al. (2002) ibid.), the above study investigated whether the identification of such mTNFα expressing cells in the mucosa could be used to identify patients who would respond to subsequent anti-TNFα therapy. Accordingly, a GMP conform, fluorescent anti-TNFα antibody (fluorescent adalimumab) was synthesized and was topically applied to the intestinal mucosa in vivo during colonoscopy in CD patients prior to adalimumab therapy. The finding from this study suggests high safety and tolerability of topically applied FITC-adalimumab in patients with CD. Endomicroscopy allowed the detection of fluorescent mTNFα expressing mucosal immune cells in CD. The in vivo and ex vivo imaging results showed that CD patients with high numbers of mTNFα expressing target cells respond significantly better to subsequent anti-TNFα therapy with adalimumab as compared to patients with low numbers of mTNFα positive mucosal target cells. These results demonstrate that in vivo and ex vivo molecular imaging with fluorescent anti-TNFα antibodies can serve as a predictive biomarker for the therapeutic response to adalimumab therapy and therefore opens new avenues for individualized therapy.

### Example 2

### Topical Administration of Anti-TNFα Inhibitor for Treatment of an Inflammatory Bowel Disease

The study in Example 1 supports the assertion that it is safe to topically deliver an anti-TNFα antibody, i.e., adalimumab, to the intestinal mucosa of patients having IBD, e.g., Crohn's disease. Thus, an anti-TNFα antibody (e.g., adalimumab), or antigen-binding portion thereof, may be delivered topically to the intestinal mucosa of a patient having an inflammatory bowel disease, such as Crohn's, for treatment. Adalimumab is administered to a subject having Crohn's disease or ulcerative colitis via a spray catheter to deliver the antibody to the intestinal mucosa. In this manner, adalimumab is delivered to the patient via local administration to the intestinal mucosa for treatment rather than through systemic administration. Efficacy for the treatment of Crohn's disease in the patient is then determined according to a decrease in the CDAI. Subsequent treatments are also performed using a spray catheter which provides for topical administration to the intestinal mucosa.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

1. A method for determining the responsiveness of a subject having inflammatory bowel disease (IBD) to treatment with a TNFα inhibitor, the method comprising
determining the level of expression of TNFα in the cells of the intestinal mucosa of the subject having IBD; and
comparing the level of expression of TNFα in the cells of the intestinal mucosa of the subject to a control level of expression of TNFα from a non-responder,
wherein a higher level of expression of TNFα in the cells of the intestinal mucosa of the subject as compared to the control level of expression of TNFα indicates that the subject will be responsive to treatment with the TNFα inhibitor, thereby predicting the responsiveness of the subject having IBD to treatment with the TNFα inhibitor.

2. A method of determining whether a TNFα inhibitor will be effective for the treatment of a subject having inflammatory bowel disease (IBD), the method comprising
determining the level of expression of TNFα in the cells of the intestinal mucosa of the subject having IBD,
wherein a higher level of expression of TNFα in the cells of the intestinal mucosa of the subject as compared to a control level of expression of TNFα for a nonresponder indicates that the TNFα inhibitor will be effective for the treatment of the subject having IBD, thereby determining whether a TNFα inhibitor will be effective for the treatment of the subject having IBD.

3. A method for treating a subject having inflammatory bowel disease (IBD), comprising
determining the level of expression of TNFα in the cells of the intestinal mucosa of the subject having IBD; and
administering a TNFα inhibitor to the subject having IBD, provided that the level of expression of TNFα in the cells of the intestinal mucosa of the subject having IBD is higher than a control level of expression of TNFα for a nonresponder, thereby treating the subject having IBD.

4. The method of any one of claims 1-3, wherein the IBD is Crohn's disease or ulcerative colitis.

5. The method of any one of claims 1-3, wherein the level of expression of membrane TNFα (mTNFα) in the cells of the intestinal mucosa of the subject having IBD is determined.

6. The method of any one of claims 1-3, wherein determining the level of expression of TNFα in the cells of the intestinal mucosa of the subject having IBD comprises topically applying a detectably labeled TNFα inhibitor to the cells of the intestinal mucosa of the subject having IBD.

7. The method of claim 6, wherein the detectably labeled TNFα inhibitor is topically applied to the cells of the intestinal mucosa of the subject having IBD during colonoscopy.

8. The method of any one of claims 1-3, wherein the level of expression of TNFα is determined using an in vivo assay.

9. The method of any one of claims 1-3, wherein the level of expression of TNFα is determined using an ex vivo assay.

10. The method of claim 9, wherein the level of expression of TNFα in the sample is determined by a technique selected from the group consisting of immunohistochemistry, immunocytochemistry, flow cytometry, ELISA and mass spectrometry.

11. The method of any one of claims 1-3, wherein the level of expression of TNFα in the sample is determined at the nucleic acid level.

12. The method of claim 11, wherein the nucleic acid level is determined using either quantitative polymerase chain reaction or expression array analysis.

13. The method of any one of claims 1-3, wherein the level of expression of TNFα is determined by confocal laser endomicroscopy.

14. The method of any one of claims 1-3, wherein the TNFα inhibitor is an anti-TNFα antibody, or antigen-binding portion thereof.

15. The method of claim 14, wherein the anti-TNFα antibody, or antigen-binding portion thereof, is selected from the group consisting of a human antibody, a chimeric antibody, and a humanized antibody.

16. The method of claim 15, wherein the chimeric anti-TNFα antibody, or antigen-binding portion thereof, is infliximab.

17. The method of claim 15, wherein the human anti-TNFα antibody, or antigen-binding portion thereof, is adalimumab or golimumab.

18. The method of claim 15, wherein the human anti-TNFα antibody, or antigen-binding portion thereof, is selected from the group consisting of
(a) an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less;
(b) an isolated human antibody comprising
a light chain variable region comprising a CDR3 domain comprising the amino acid sequence of SEQ ID NO: 3, a CDR2 domain comprising the amino acid sequence of SEQ ID NO: 5, and a CDR1 domain comprising the amino acid sequence of SEQ ID NO: 7, and
a heavy chain variable region comprising a CDR3 domain comprising the amino acid sequence of SEQ ID NO: 4, a CDR2 domain comprising the amino acid sequence of SEQ ID NO: 6, and a CDR1 domain comprising the amino acid sequence of SEQ ID NO: 8, and
(c) an isolated human antibody comprising a light chain variable region (LCVR) comprising the amino acid sequence of SEQ ID NO: 1 and a heavy chain variable region (HCVR) comprising the amino acid sequence of SEQ ID NO: 2.

19-20. (canceled)

21. The method of any one of claims 1-3, wherein the method determines or predicts clinical responsiveness in the subject.

22. A method for treating a subject having inflammatory bowel disease (IBD), the method comprising
selecting a subject having IBD and having a level of expression of TNFα in the intestinal mucosa which is higher than a control level of expression of TNFα from a nonresponder; and
topically administering a TNFα inhibitor to the intestinal mucosa of the subject having IBD, thereby treating the subject having IBD.

23. The method of claim 22, wherein the IBD is Crohn's disease or ulcerative colitis.

24. The method of claim 22, wherein the TNFα inhibitor is administered using a spraying catheter.

25. The method of claim 22, wherein the level of expression of membrane TNFα (mTNFα) in the cells of the intestinal mucosa of the subject having IBD is determined.

26. The method of claim 22, wherein the level of expression of TNFα is determined using an in vivo assay or an ex vivo assay.

27. The method of claim 22, wherein the TNFα inhibitor is an anti-TNFα antibody, or antigen-binding portion thereof.

28. The method of claim 27, wherein the anti-TNFα antibody, or antigen-binding portion thereof, is selected from the group consisting of a human antibody, a chimeric antibody, and a humanized antibody.

29. The method of claim 28, wherein the chimeric anti-TNFα antibody, or antigen-binding portion thereof, is infliximab.

30. The method of claim 28, wherein the human anti-TNFα antibody, or antigen-binding portion thereof, is adalimumab or golimumab.

31. The method of claim 28, wherein the human anti-TNFα antibody, or antigen-binding portion thereof, is
(a) an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less;
(b) an isolated human antibody comprising
a light chain variable region comprising a CDR3 domain comprising the amino acid sequence of SEQ ID NO: 3, a CDR2 domain comprising the amino acid sequence of SEQ ID NO: 5, and a CDR1 domain comprising the amino acid sequence of SEQ ID NO: 7, and
a heavy chain variable region comprising a CDR3 domain comprising the amino acid sequence of SEQ ID NO: 4, a CDR2 domain comprising the amino acid sequence of SEQ ID NO: 6, and a CDR1 domain comprising the amino acid sequence of SEQ ID NO: 8, and
(c) an isolated human antibody comprising a light chain variable region (LCVR) comprising the amino acid sequence of SEQ ID NO: 1 and a heavy chain variable region (HCVR) comprising the amino acid sequence of SEQ ID NO: 2.

32-33. (canceled)

34. A kit for determining if a TNFα inhibitor will be effective for the treatment of a subject having inflammatory bowel disease (IBD), the kit comprising
a means for determining the level of expression of TNFα in the cells of the intestinal mucosa of the subject having IBD, and
instructions for recommended treatment for the subject based on the level of expression of TNFα in the cells of the intestinal mucosa of the subject having IBD,
wherein a higher level of expression of TNFα in the cells of the intestinal mucosa of the subject as compared to a control level of expression of TNFα from a nonresponder indicates that the TNFα inhibitor will be effective for the treatment of the subject having IBD.

35. The kit of claim 34, further comprising a pharmaceutical composition comprising the TNFα inhibitor.

36. The kit of claim 34, wherein the means for determining the level of expression of TNFα in the cells of the intestinal mucosa of the subject having IBD comprises a detectably labeled anti-TNFα antibody, or antigen-binding portion thereof.

37. The kit of claim 36, wherein the detectably labeled anti-TNFα antibody, or antigen-binding portion thereof, is labeled with fluorescein isothiocyanate (FITC).

38. The kit of claim 36, wherein the anti-TNFα antibody, or antigen-binding portion thereof, is infliximab.

39. The kit of claim 36, wherein the anti-TNFα antibody, or antigen-binding portion thereof, is adalimumab or golimumab.

40. The kit of claim 36, wherein the anti-TNFα antibody, or antigen-binding portion thereof, is
(a) an isolated human antibody that dissociates from human TNFα with a K_{d} of 1×10⁻⁸ M or less and a k_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less;
(b) an isolated human antibody comprising
a light chain variable region comprising a CDR3 domain comprising the amino acid sequence of SEQ ID NO: 3, a CDR2 domain comprising the amino acid sequence of SEQ ID NO: 5, and a CDR1 domain comprising the amino acid sequence of SEQ ID NO: 7, and a heavy chain variable region comprising a CDR3 domain comprising the amino acid sequence of SEQ ID NO: 4, a CDR2 domain comprising the amino acid sequence of SEQ ID NO: 6, and a CDR1 domain comprising the amino acid sequence of SEQ ID NO: 8, and
(c) an isolated human antibody comprising a light chain variable region (LCVR) comprising the amino acid sequence of SEQ ID NO: 1 and a heavy chain variable region (HCVR) comprising the amino acid sequence of SEQ ID NO: 2.

41-42. (canceled)

43. The kit of claim 34, wherein the IBD is Crohn's disease or ulcerative colitis.

44. The kit of claim 34, wherein the means for determining the level of expression of TNFα in the cells is a means for determining the level of membrane TNFα in the cells of the intestinal mucosa.


SEQUENCE LISTING (38 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  adalimumab light chain variable region CDR3
QRYNRAPYX

SEQ ID NO: 4 - protein, 12 aa, Artificial Sequence
  adalimumab heavy chain variable region CDR3
VSYLSTASSLDX

SEQ ID NO: 5 - protein, 7 aa, Artificial Sequence
  adalimumab light chain variable region CDR2
AASTLQS

SEQ ID NO: 6 - protein, 17 aa, Artificial Sequence
  adalimumab heavy chain variable region CDR2
AITWNSGHIDYADSVEG

SEQ ID NO: 7 - protein, 11 aa, Artificial Sequence
  adalimumab light chain variable region CDR1
RASQGIRNYLA

SEQ ID NO: 8 - protein, 5 aa, Artificial Sequence
  adalimumab heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt

SEQ ID NO: 38 - protein, 15 aa, Artificial Sequence
  flexible linker
GGGGSGGGGSGGGGS